(19) United States
(12) Patent Application Publication
(10) Pub. No.: US 2022/0339176 A1
(43) Pub. Date: Oct. 27, 2022

(54) Methods of Treatments to Prolong Gestation and Complications of Menstruation or Gestation

(71) Applicant: The Board of Trustees of the Leland Stanford Junior University, Stanford, CA (US)

(72) Inventors: Liang Liang, Palo Alto, CA (US); Michael P. Snyder, Stanford, CA (US)

(73) Assignee: The Board of Trustees of the Leland Stanford Junior University, Stanford, CA (US)

(21) Appl. No.: 17/754,102

(22) PCT Filed: Sep. 23, 2020
(86) PCT No.: PCT/US2020/052311

§ 371 Date: Mar. 23, 2022

Related U.S. Application Data
(60) Provisional application No. 62/904,615, filed on Sep. 23, 2019.

(51) Int. Cl. A61K 31/704; A61K 31/573; A61K 31/566; A61K 31/5685; A61K 31/7008; A61K 31/4406; A61K 31/685; A61P 15/06

(52) U.S. Cl. CPC A61K 31/704; A61K 31/573; A61K 31/566; A61K 31/5685; A61K 31/7008; A61K 31/4406; A61K 31/685; A61P 15/06

(57) ABSTRACT

Methods of treatment for menstrual complications, gestational complications, and to prolong gestation are described. Treatments include administration of a compound related to regulation of gestational progress or uterine contractions.

### CROSS REFERENCE TO RELATED APPLICATIONS

This application claims priority to U.S. Provisional Application Ser. No. 62/904,615, entitled “Methods of Treatments for Gestational Complications,” filed Sep. 23, 2019, which is incorporated herein by reference in its entirety.

### TECHNICAL FIELD

The invention is generally directed toward methods of assessment and treatment for menstrual complications including menorrhagia and dysmenorrhea and gestational complications including spontaneous preterm labor, spontaneous abortion, early term birth, recurrent preterm birth, recurrent early term birth, and recurrent pregnancy loss.

### BACKGROUND

Various menstrual complications include menorrhagia and dysmenorrhea. Each of these disorders is related to uterine wall lining (i.e., the endometrium) and uterine contractions. Menorrhagia is heavy and/or prolonged bleeding related to menstruation, which may arise from various sources including an imbalance of hormones, uterine fibroids, contraceptive devices and medications, ovary dysfunction, polyps, and other conditions related to life changes (e.g., induced stress). Dysmenorrhea is throbbing or cramping pain related to menstruation, which may arise due to hormone imbalance, uterine fibroids, or endometriosis.

Various gestational complications include spontaneous preterm labor, spontaneous abortion, early term birth, recurrent preterm birth, recurrent early term birth, and recurrent pregnancy loss. Each of these disorders is related to premature uterine contractions during pregnancy. Spontaneous preterm labor is the opening of the cervix after week 20 and before week 37 of gestation, and can result in a preterm birth that can dramatically hinder the health and may result in death of the newborn and/or mother. Spontaneous abortion (also referred to as miscarriage) is the spontaneous loss of a pregnancy before week 20 of gestation. Early term birth is the opening of the cervix after week 37 and before week 39 of gestation, and can result in a birth that less desirable than a full term birth of greater than 39 weeks of gestation. Recurrent preterm birth is a condition in which a woman experiences two or more pregnancies that go into labor prior to week 37 of gestation. Recurrent early term birth is a condition in which a woman experiences two or more pregnancies that go into labor prior to week 39 of gestation. Recurrent pregnancy loss is a condition in which a woman experiences two or more spontaneous losses of pregnancy.

Progesterone and 17-α-hydroxyprogesterone, and derivatives thereof, are utilized as therapeutic in menorrhagia, dysmenorrhea, spontaneous preterm labor, spontaneous abortion, recurrent preterm birth, early term birth, and recurrent pregnancy loss. Unfortunately, progesterone and the derivatives are at only moderately effective in many of these disorders. Progesterone and 17-α-hydroxyprogesterone derivatives include progestins and 17-α-hydroxyprogesterone caproate.

### SUMMARY

Various embodiments are directed towards methods for assessing and treating menstrual complications and gestational complications. In various embodiments, an individual having a menstrual complication is administered a metabolic compound or derivative, which can be provided to mitigate and/or alleviate the menstrual complication. In various embodiments, a pregnant individual is administered a metabolic compound or derivative, which can be provided to prolong gestation. In various embodiments, a pregnant individual having a gestational complication is administered a metabolic compound or derivative, which can be provided to mitigate and/or alleviate the gestational complication.

In an embodiment, a pregnant individual is treated for recurrent preterm birth, recurrent early term birth, or recurrent pregnancy loss. A pregnant individual is determined to have been diagnosed with recurrent preterm birth, recurrent early term birth, or recurrent pregnancy loss. The individual is monitored during the individual's gestation. The individual is administered at least one compound to mitigate early term birth, preterm birth or pregnancy loss. The at least one compound is estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, dehydroisoandrosterone sulfate (DHEA-S) or an alternative steroidal compound thereof or a derivative thereof or a metabolite within the synthesis pathway thereof, estrone 3-sulfate, N-Acetyl-D-glucosamine, 3-acetoxypyridine, 5-pregnane-3,7-diol-20-one-3-sulfate, androsterone, PC(22:1/22:1) (Lecithin), LPC(20:5), 7-methylguanine, androsterone sulfate, PE(P-16:0e/0:0) (LysoPE(P-16:0/0:0), 1-(1Z-hexadecenyl)-sn-glycero-3-phosphoethanolamine, or pregnenolone sulfate.

In another embodiment, at least two of the following compounds are administered to the individual: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, dehydroisoandrosterone sulfate (DHEA-S) or an alternative steroidal compound thereof or a derivative thereof or a metabolite within the synthesis pathway thereof, estrone 3-sulfate, N-Acetyl-D-glucosamine, 3-acetoxypyridine, 5-pregnane-3,7-diol-20-one-3-sulfate, androsterone, PC(22:1/22:1) (Lecithin), LPC(20:5), 7-methylguanine, androsterone sulfate, PE(P-16:0e/0:0) (LysoPE(P-16:0/0:0), 1-(1Z-hexadecenyl)-sn-glycero-3-phosphoethanolamine, or pregnenolone sulfate.

In yet another embodiment, the individual is further administered progesterone, 17-α-hydroxyprogesterone, 17-α-hydroxyprogesterone caproate, or a progestin.

In a further embodiment, a biological sample is extracted from the individual. It is determined that the individual has deficiency of at least one of estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, or pregnenolone sulfate. The compound that is administered to the individual is the at least one deficient metabolite, an alternative steroidal compound of the at least one deficient metabolite, a derivative of the at least one deficient metabolite, or a metabolite within the synthesis pathway the at least one deficient metabolite.

In still yet another embodiment, a pregnant individual is treated for early term birth, spontaneous preterm birth or spontaneous abortion. It is determined that a pregnant individual is experiencing early term birth, spontaneous preterm birth or spontaneous abortion. The individual is administered at least one tocolytic compound to mitigate uterine contractions. The at least one compound is estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

In yet a further embodiment, at least two of the following tocolytic compounds are administered to the individual: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

In an even further embodiment, the individual is further administered progesterone, 17-α-hydroxyprogesterone, 17-α-hydroxyprogesterone caproate, or a progestin to the individual.

In yet an even further embodiment, a biological sample is extracted from the individual. It is determined that the individual has deficiency of at least one of estriol-16-glucuronide, tetrahydrodeoxycorticosterone (THDOC), androstane-3,17-diol, or dehydroisoandrosterone sulfate (DHEA-S). The compound that is administered to the individual is the at least one deficient metabolite, an alternative steroidal compound of the at least one deficient metabolite, a derivative of the at least one deficient metabolite, or a metabolite within the synthesis pathway the at least one deficient metabolite.

In still yet an even further embodiment, an individual is treated for menorrhagia or dysmenorrhea. It is determined that the individual is diagnosed with menorrhagia or dysmenorrhea. The individual is administered at least one compound to mitigate menorrhagia or dysmenorrhea. The at least one compound is estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

In still yet an even further embodiment, at least two of the following compounds are administered to the individual estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

In still yet an even further embodiment, the individual is further administered progesterone, 17-α-hydroxyprogesterone, 17-α-hydroxyprogesterone caproate, or a progestin to the individual.

In still yet an even further embodiment, a biological sample is extracted from the individual. It is determined that the individual has deficiency of at least one of estriol-16-glucuronide, tetrahydrodeoxycorticosterone (THDOC), androstane-3,17-diol, or dehydroisoandrosterone sulfate (DHEA-S). The compound that is administered to the individual is the at least one deficient metabolite, an alternative steroidal compound of the at least one deficient metabolite, a derivative of the at least one deficient metabolite, or a metabolite within the synthesis pathway the at least one deficient metabolite.

In still yet an even further embodiment, a pregnant individual is treated to prolong gestation. An individual is determined to be pregnant. Prior to the individual having uterine contractions associated with neonatal delivery, the individual is administered at least one compound to prolong gestation. The at least one compound is estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

In still yet an even further embodiment, at least two of the following compounds are administered to the individual estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

In still yet an even further embodiment, the individual is further administered progesterone, 17-α-hydroxyprogesterone, 17-α-hydroxyprogesterone caproate, or a progestin to the individual.

In still yet an even further embodiment, the individual is generally healthy or has no known medical issues related to gestation.

In still yet an even further embodiment is a medicament for use in mitigating uterine contractions in an individual. The medicament includes estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

In still yet an even further embodiment, the medicament comprises at least two of the following compounds estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

In still yet an even further embodiment, the alternative steroidal compound of estriol-16-glucuronide is estradiol 17β-D-glucuronide.

In still yet an even further embodiment, the alternative steroidal compound of tetrahydrodeoxycorticosterone (THDOC) is 5α-dihydrodeoxycorticosterone (DHDOC).

In still yet an even further embodiment, the alternative steroidal compound of androstane-3,17-diol is oxandrolone, oxymetholone, stanozolol, norethandrolone, quinbolone, metandienone metenolone, prasterone, or stanolone.

In still yet an even further embodiment, the derivative of androstane-3,17-diol is 17α-ethynyl-3α-androstanediol (apoptone), 17α-ethynyl-3β-androstanediol, 17α-ethynyl-5-androstenediol, 17α-ethynyl-5-androstenediol 3β-cyclohexanepropionate, 17α-ethynylestradiol, 17α-ethynyltestosterone, or 17α-ethynyldihydrotestosterone.

In still yet an even further embodiment, the derivative of androstane-3,17-diol has a structural formula:

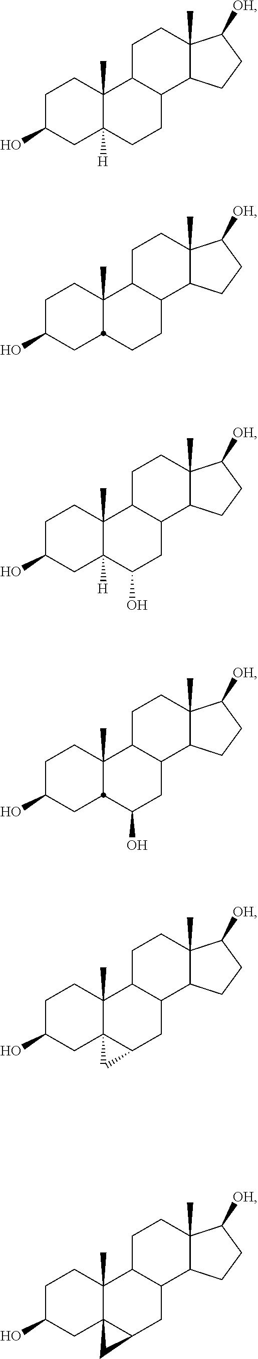

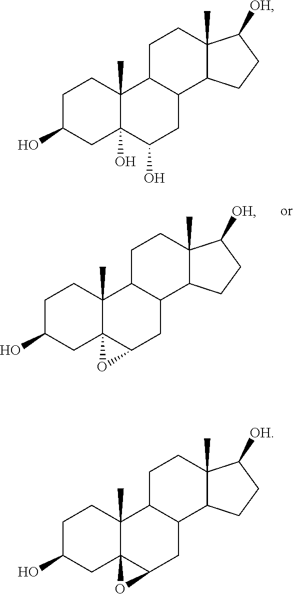 [SMILES: C[C@]12CCC3C(C[C@H](O)[C@@]4(O)C[C@@H](O)CC[C@]34C)C1CC[C@@H]2O ; C[C@]12CCC3C(C[C@@H]4O[C@@]45C[C@@H](O)CC[C@]35C)C1CC[C@@H]2O ; C[C@]12CCC3C(C[C@H]4O[C@]45C[C@@H](O)CC[C@]35C)C1CC[C@@H]2O]

In still yet an even further embodiment, the androstane-3,17-diol or derivative thereof has a structural formula:

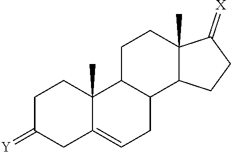

X and Y are each independently: O, NR, NOR, NNR¹R², OR⁴ α/R³ α, OR⁴ β/R³ β, —O(CH₂)ₙO—, or —O(CHR)ₙO—.
R, R², R³, and R⁴ are each independently: H, alkyl, alkenyl, alkynyl, aryl, or heteroaryl.
n is 2, 3, or 4.

In still yet an even further embodiment, the metabolite within the synthesis pathway of androstane-3,17-diol is dehydroisoandrosterone sulfate (DHEA-S), 4-androstene-3-17-dione (androstenedione; 4A), testosterone, 5α-dihydrotestosterone (5α-DHT), 5β-dihydrotestosterone (5β-DHT), DHEA (dehydroepiandrosterone), or androsterone.

In still yet an even further embodiment, the alternative steroidal compound dehydroisoandrosterone sulfate (DHEA-S) is 7α-hydroxy-DHEA, 16α-hydroxy-DHEA, 17α-hydroxypregnenolone, norethandrolone, oxandrolone, quinbolone, oxymetholone, metenolone, metandienone, stanozolol, and stanolone.

In still yet an even further embodiment, the derivative of dehydroisoandrosterone sulfate (DHEA-S) is 3β-dehydroxy-16α-fluoro-DHEA (fluasterone).

In still yet an even further embodiment, the derivative of dehydroisoandrosterone sulfate (DHEA-S) has a structural formula:

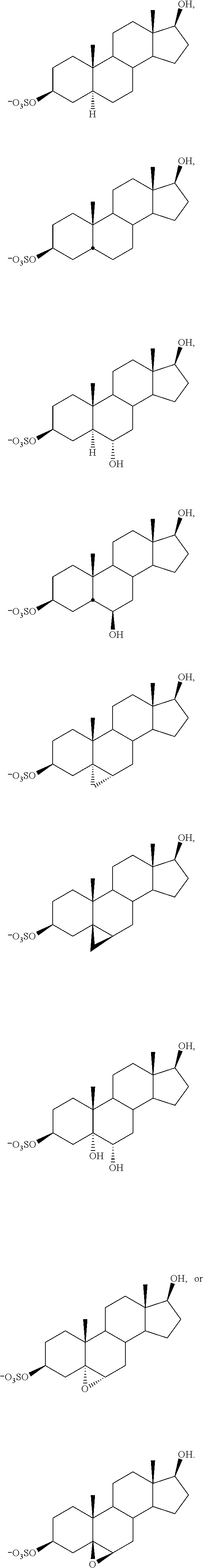

In still yet an even further embodiment, the dehydroisoandrosterone sulfate (DHEA-S) or derivative thereof has a structural formula:

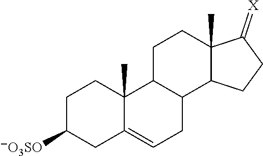

X is O, NR, NOR, NNR¹R², OR⁴ α/R³ α, OR⁴ β/R³ β, —O(CH₂)ₙO—, or —O(CHR)ₙO—.
R, R², R³, and R⁴ are each independently: H, alkyl, alkenyl, alkynyl, aryl, or heteroaryl.
n is 2, 3, or 4.

In still yet an even further embodiment, the metabolite within the synthesis pathway of dehydroisoandrosterone sulfate (DHEA-S) is 4-androstene-3-17-dione (androstenedione; 4A), testosterone, 5α-dihydrotestosterone (5α-DHT), 5β-dihydrotestosterone (5β-DHT), DHEA (dehydroepiandrosterone), androsterone, and androstane-3,17-diol.

In still yet an even further embodiment, the medicament further includes progesterone, 17-α-hydroxyprogesterone, 17-α-hydroxyprogesterone caproate, or a progestin to the individual.

In still yet an even further embodiment, the medicament is for treating recurrent preterm birth, recurrent early term birth, or recurrent pregnancy loss.

In still yet an even further embodiment, the medicament is a tocolytic for treating early term birth, spontaneous preterm birth, or spontaneous abortion in a pregnant individual.

In still yet an even further embodiment, the medicament is for treating menorrhagia or dysmenorrhea.

In still yet an even further embodiment, the medicament is for prolonging gestation of a pregnant individual.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a mutual correlation network of metabolites with progesterone in blood during human gestation, utilized in accordance with various embodiments.

FIG. 2 provides charts plotting the relative concentrations of steroid hormones and phospholipids and DHEA-S progressing through human gestation, utilized in accordance with various embodiments.

FIG. 3 provides a chart of closeness ranking of metabolites correlating with gestational age, utilized in accordance with various embodiments.

FIG. 4A provides images of a collagen contraction assay with human uterine smooth muscle cells treated with various compounds, utilized in accordance with various embodiments.

FIG. 4B provides graphical results of a collagen contraction assay with human uterine smooth muscle cells treated with various compounds, utilized in accordance with various embodiments.

FIG. 5A provides graphical results of a myography assay with murine uterine muscle tissue treated with various compounds, utilized in accordance with various embodiments.

FIGS. 5B and 5C provide graphical results of a myography assay with murine uterine muscle tissue stimulated with oxytocin and treated with androstane-3,17-diol, utilized in accordance with various embodiments.

FIG. 5D provides representative myography readings of murine uterine muscle tissue contractions stimulated with oxytocin and treated with androstane-3,17-diol, utilized in accordance with various embodiments.

FIG. 5E provides representative myography readings of murine uterine muscle tissue contractions treated with various compounds, utilized in accordance with various embodiments.

FIGS. 5F to 5H provide graphical results of a myography assay with murine uterine muscle tissue stimulated with oxytocin and treated with: progesterone, androstane-3,17-diol, progesterone+androstane-3-17-diol, utilized in accordance with various embodiments.

FIG. 6A provides a schematic of an experimental design of treating a murine model of moderate preterm delivery with various compounds, utilized in accordance with various embodiments.

FIG. 6B provides graphical results of treating a murine model of moderate preterm delivery with various compounds, utilized in accordance with various embodiments.

FIG. 7 provides a schematic of an experimental design of treating a murine model of severe preterm delivery with androstane-3,17-diol, utilized in accordance with various embodiments.

FIG. 8A provides a schematic of an experimental design of treating a murine model of severe preterm delivery with tocolytic androstane-3,17-diol, utilized in accordance with various embodiments.

FIG. 8B provides graphical results of treating healthy mice or a murine model of severe preterm delivery with tocolytic androstane-3,17-diol, utilized in accordance with various embodiments.

### DETAILED DESCRIPTION

Turning now to the drawings and data, methods to treat an individual having a menstrual complication, a gestational complication, or to prolong gestation in accordance with various embodiments are described. In some embodiments, an individual is administered a compound that regulates uterine contraction. In some embodiments, an individual having a menstrual complication, such as (for example) cramping, dysmenorrhea, and menorrhagia is administered a compound described herein to treat the menstrual complication. In some embodiments, an individual having a gestational complication, such as (for example) spontaneous preterm labor, spontaneous abortion, recurrent preterm birth, early term birth, or recurrent pregnancy loss is administered a compound described herein to treat the gestational complication. In some embodiments, gestational hormone levels are measured and if in an imbalance, a treatment of a compound is administered.

Progesterone and 17-α-hydroxyprogesterone, and derivatives thereof, have been utilized in various treatments for menstrual and gestational complications, including (but not limited to) cramping, dysmenorrhea, spontaneous preterm labor, spontaneous abortion, recurrent preterm birth, early term birth, and recurrent pregnancy loss. It is now understood that various metabolites and hormones described herein are involved in regulating gestational progress similar to or better than progesterone. Further, data has been generated to show that various compounds can be utilized to regulate uterine contractions, time to delivery, and/or gestational progress, and in many cases better than the current standard of progesterone treatments. Accordingly, various compounds can be utilized in lieu of or in addition to progesterone and 17-α-hydroxyprogesterone, and derivatives thereof to treat a menstrual and/or gestational complication.

### Compounds

Several embodiments are directed towards compounds and their use as therapeutics to treat an individual having a menstrual complication, a gestational complication, or to prolong gestation. A number of compounds have been identified that that reduce uterine cell contractions and thus can be utilized to mitigate complications that arise from premature and/or abnormal uterine contractions. In some embodiments, a compound is a metabolite. In some embodiments, a compound is a steroidal hormone. In some embodiments, a compound is a derivative of a metabolite or steroidal hormone.

In a number of embodiments, a compound to be administered is estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, pregnenolone sulfate, a derivative thereof, or a combination thereof. In some embodiments, progesterone, 17-α-hydroxyprogesterone or a derivative thereof is also administered. Progesterone and 17-α-hydroxyprogesterone derivatives include (but are not limited to) progestins and 17-α-hydroxyprogesterone caproate.

In some embodiments, the compound to be administered is androstane-3,17-diol or a derivative thereof. Androstane-3,17-diol structural formula is as follows:

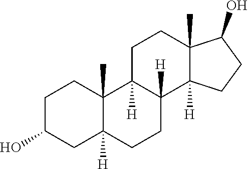

Various embodiments utilize various stereoisomers of androstane-3,17-diol including (but not limited to) androstane-3α,17α-diol, androstane-3α,17β-diol, androstane-3β,17α-diol, androstane-3β,17β-diol, and combinations thereof.

Derivatives of androstane-3,17-diol include (but are not limited to) 17α-ethynyl-3α-androstanediol (apoptone), 17α-ethynyl-3β-androstanediol, 17α-ethynyl-5-androstenediol, 17α-ethynyl-5-androstenediol 3β-cyclohexanepropionate, 17α-ethynylestradiol, 17α-ethynyltestosterone, and 17α-ethynyldihydrotestosterone. Derivatives of androstane-3,17-diol further include (but are not limited to) the following structural formulae:

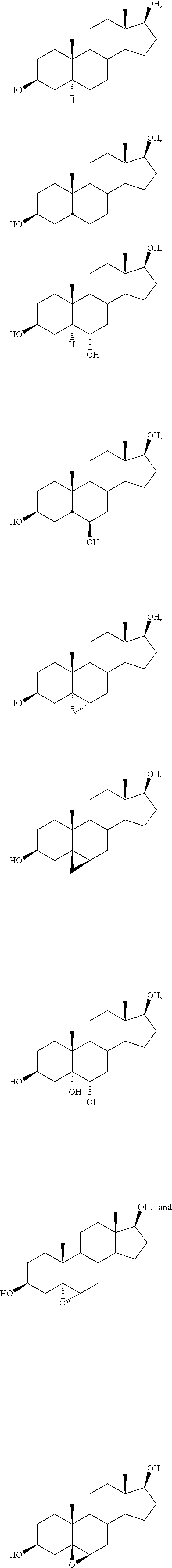

In some embodiments, androstane-3,17-diol and/or derivatives to be utilized include the following structural formulae:

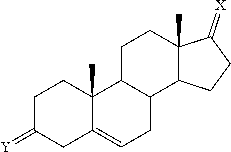

X and Y are each independently: O, NR, NOR, NNR¹R², OR⁴ α/R³ α, OR⁴ β/R³ β, —O(CH₂)ₙO—, or —O(CHR)ₙO—.
R, R², R³, and R⁴ are each independently: H, alkyl, alkenyl, alkynyl, aryl, or heteroaryl.
n is 2, 3, or 4.

In some embodiments, the compound to be utilized is a metabolite within the synthesis pathway of androstane-3,17-diol. Metabolites within the synthesis pathway of androstane-3,17-diol include (but are not limited to) dehydroisoandrosterone sulfate (DHEA-S), 4-androstene-3-17-dione (androstenedione; 4A), testosterone, 5α-dihydrotestosterone (5α-DHT), 5β-dihydrotestosterone (5β-DHT), DHEA (dehydroepiandrosterone), and androsterone. In some embodiments, a metabolite within the synthesis pathway of androstane-3,17-diol is utilized as a prodrug.

In some embodiments, the compound to be administered is an alternative steroidal compound of androstane-3,17-diol. Alternative steroidal compounds for androstane-3,17-diol include (but are not limited to) oxandrolone, oxymetholone, stanozolol, norethandrolone, quinbolone, metandienone metenolone, prasterone, stanolone.

In some embodiments, the compound to be delivered is dehydroisoandrosterone sulfate (DHEA-S) or a derivative thereof. DHEA-S structural formula is as follows:

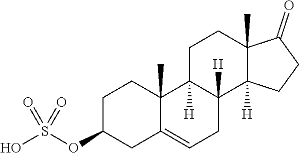

Various embodiments utilize various stereoisomers of DHEA-S including (but not limited to) (3α, 21α), (3α, 21β), (3β, 21α), (3β, 21β), and combinations thereof.

Derivatives of DHEA-S include (but are not limited to) 3β-dehydroxy-16α-fluoro-DHEA (fluasterone). Derivatives of DHEA-S further include (but are not limited to) the following structural formulae:

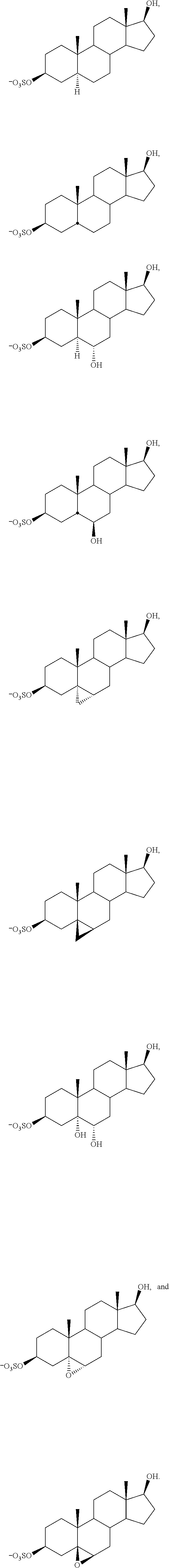

In some embodiments, DHEA-S and/or derivatives to be utilized include the following structural formulae:

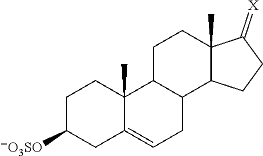

X is: O, NR, NOR, NNR¹R², OR⁴ α/R³ α, OR⁴ β/R³ β, —O(CH₂)ₙO—, or —O(CHR)ₙO—.
R, R², R³, and R⁴ are each independently: H, alkyl, alkenyl, alkynyl, aryl, or heteroaryl.
n is 2, 3, or 4.

In some embodiments, the compound to be utilized is a metabolite within the synthesis pathway of DHEA-S. Metabolites within the synthesis pathway of DHEA-S include (but are not limited to), 4-androstene-3-17-dione (androstenedione; 4A), testosterone, 5α-dihydrotestosterone (5α-DHT), 5β-dihydrotestosterone (5β-DHT), DHEA (dehydroepiandrosterone), androsterone, and androstane-3,17-diol. In some embodiments, a metabolite within the synthesis pathway of DHEA-S is utilized as a prodrug.

In some embodiments, the compound to be administered is an alternative steroidal compound of DHEA-S. Alternative steroidal compounds for DHEA-S include (but are not limited to) 7α-hydroxy-DHEA, 16α-hydroxy-DHEA, 17α-hydroxypregnenolone, norethandrolone, oxandrolone, quinbolone, oxymetholone, metenolone, metandienone, stanozolol, and stanolone.

In some embodiments, the compound to be administered is estriol-16-glucuronide or a derivative thereof. In some embodiments, the compound to be administered is an alternative steroidal compound of estriol-16-glucuronide. Estriol-16-glucoronide structural formula is as follows:

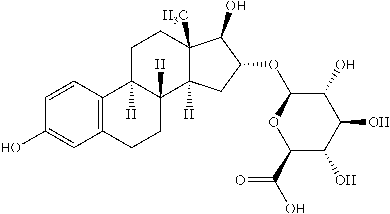

Various embodiments utilize various stereoisomers of estriol-16-glucuronide including (but not limited to estriol-16α-(β-D-glucuronide) andestriol-16-β-(α-D-glucuronide), and combinations thereof. Alternative steroidal compounds for estriol-16-glucuronide include (but are not limited to) estriol and estradiol 17β-D-glucuronide.

In some embodiments, the compound to be administered is tetrahydrodeoxycorticosterone (THDOC) or a derivative thereof. THDOC structural formula is as follows:

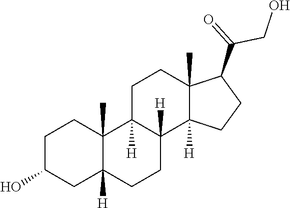

Various embodiments utilize various stereoisomers of THDOC including (but not limited to (3α, 21α), (3α, 21β), (3β, 21α), (3β, 21β), and combinations thereof. In some embodiments, the compound to be administered is an alternative steroidal compound of THDOC. Alternative steroidal compounds for THDOC include (but are not limited to) 5α-dihydrodeoxycorticosterone (DHDOC).

### Pharmaceutical Formulae

Provided herein are various embodiments of pharmaceuticals for use in a treatment of menstrual complications, gestational complications, and/or to prolong gestation, together with one or more pharmaceutically acceptable carriers thereof and optionally one or more other active ingredients. Proper formulation is dependent upon the route of administration chosen. Any of the well-known techniques, carriers, and excipients may be used as suitable and as understood in the art. Pharmaceutical compositions may be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared utilizing the various method embodiments as described herein.

The term “active ingredient” refers to a compound, which is administered, alone or in combination with one or more pharmaceutically acceptable excipients or carriers, to a subject for treating, preventing, or ameliorating one or more symptoms of a disorder. In various embodiments, active ingredients include estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, pregnenolone sulfate, progesterone, and 17-α-hydroxyprogesterone, and derivatives thereof.

The compounds disclosed herein can exist as therapeutically acceptable salts.

The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed., (Wiley-VCH and VHCA, Zurich, 2002) and Berge et al, J. Pharm. Sci. 1977, 66, 1-19.

Numerous coating agents can be used in accordance with various embodiments of the invention. In some embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

Various embodiments of formulations also include at least one disintegrating agent. In some embodiments, a disintegrating agent is a super disintegrant agent. In many embodiments, disintegrants are combined with a resin. Additional disintegrating agents include, but are not limited to, agar, calcium carbonate, maize starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

Several embodiments of a formulation further utilize other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulation of the present invention include, but are not limited to, maltol, vanillin, ethyl vanillin, menthol, citric acid, fumaric acid, ethyl maltol and tartaric acid.

Multiple embodiments of a formulation also include a surfactant. In certain embodiments, surfactants are selected from the group consisting of Tween 80, sodium lauryl sulfate, and docusate sodium.

Various embodiments of a formulation also include a lubricant. In certain embodiments, lubricants are selected from the group consisting of, but are not limited to, magnesium stearate, stearic acid, sodium stearyl fumarate, calcium stearate, hydrogenated vegetable oil, mineral oil, fish oil, castor oil, sesame oil, polyethylene glycol, polyethylene glycol 4000-6000, talc, and glyceryl behenate.

Modes of administration, in accordance with multiple embodiments, include, but are not limited to, oral, intravenous, subcutaneous, intramuscular, intrauterine, intraperitoneal, or transmucosal (e.g., sublingual, nasal, vaginal or rectal). The actual amount of drug needed will depend on factors such as the size, age and severity of disease in the afflicted individual. The actual amount of drug needed will also depend on the effective concentration ranges of the various active ingredients. Vehicles of administration, in accordance with various embodiments, include ointments, solutions, gels, creams, suppositories, implants, tablets, or capsules, as appropriate.

In some embodiments, active ingredients are administered in a therapeutically effective amount as part of a course of treatment. As used in this context, to “treat” means to ameliorate at least one symptom of a disorder to be treated or to provide a beneficial physiological effect. For example, one such amelioration of a symptom could be reduction of risk of spontaneous preterm labor, spontaneous abortion, recurrent preterm birth, early term birth, or recurrent pregnancy loss.

A therapeutically effective amount can be an amount sufficient to prevent reduce, ameliorate or eliminate the symptoms of gestational complications susceptible to such treatment.

Dosage, toxicity and therapeutic efficacy of the compounds can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to other tissue and organs and, thereby, reduce side effects.

Data obtained from cell culture assays or animal studies can be used in formulating a range of dosage for use in humans. If the pharmaceutical is provided systemically, the dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration or within the local environment to be treated in a range that includes the ED₅₀ as determined in cell culture or animal models. Such information can be used to more accurately determine useful doses in humans. Levels in plasma may be measured, for example, by mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day, as determined to be beneficial. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result.

Preservatives and other additives, like antimicrobial, antioxidant, chelating agents, and inert gases, can also be present. One common preservative is benzyl alcohol. (See generally, Remington: The Science and Practice of Pharmacy, 21st Edition, Lippincott Williams & Wilkins: Philadelphia, Pa., 2005.)

### Methods of Treatment

Several embodiments are directed towards treatments of individuals with metabolic compounds or derivatives thereof for menstrual complications, gestational complications, or to prolong pregnancy. In some embodiments, metabolic compounds or derivatives thereof are administered to an individual having menorrhagia or dysmenorrhea. In some embodiments, metabolic compounds or derivatives thereof are administered to an individual experiencing spontaneous preterm labor, early term labor or spontaneous abortion. In some embodiments, metabolic compounds or derivatives thereof are administered to an individual that have a history of recurrent preterm birth or recurrent pregnancy loss. In some embodiments, metabolic compounds or derivatives thereof are administered to an individual having a deficiency of the metabolic compound in relation to gestational progress. In some embodiments, metabolic compounds or derivatives thereof are administered to an individual to prolong their gestation.

Menorrhagia is having and/or prolonged bleeding related to menstruation. Dysmenorrhea is throbbing or cramping pain related to menstruation. Each of these conditions are related to uterine wall contractions during the menstrual cycle. Further, newly acquired data demonstrates that various metabolic compounds are involved in preventing uterine wall contractions and thus can be utilized in as part of treatment to mitigate menorrhagia and/or dysmenorrhea. Accordingly, in some embodiments, when an individual is determined to have menorrhagia and/or dysmenorrhea, the individual is administered estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, pregnenolone sulfate, a derivative thereof, or a combination thereof. In some embodiments the individual is administered an alternative steroidal compound of estriol-16-glucuronide, THDOC, androstane-3,17-diol, DHEA-S, a derivative thereof, or a combination thereof. In some embodiments, the individual is additionally administered progesterone, 17-α-hydroxyprogesterone, or a derivative thereof.

Administration of compounds described herein can be combined with standards of care for the complication. When an individual has menorrhagia, in some embodiments, an individual is additionally administered a compound in combination with a nonsteroidal anti-inflammatory drug (NSAID), an oral contraceptive, progesterone, 17-α-hydroxyprogesterone, progestin (via oral pill, transdermal patch, or hormone releasing intrauterine device), or a combination thereof. When an individual has dysmenorrhea, in some embodiments, an individual is additionally administered a compound in combination with surgery, a nonsteroidal anti-inflammatory drug (NSAID), an oral contraceptive, progesterone, 17-α-hydroxyprogesterone, a progestin (via oral pill, transdermal patch, or hormone releasing intrauterine device) or a combination thereof.

Spontaneous preterm labor is the opening of the cervix after week 20 and before week 37 of gestation. Early term birth is the opening of the cervix between 37 weeks, 0 days and 38 weeks, 6 days. Spontaneous abortion is the spontaneous loss of a pregnancy before week 20 of gestation. Each of these conditions are related to uterine wall contractions occurring prematurely during gestational progress before reaching full-term. Further, newly acquired data demonstrates that various metabolic compounds are involved in maintaining proper gestational time course and preventing uterine wall contractions and thus can be utilized in as part of treatment to mitigate spontaneous preterm and early term labor and/or spontaneous abortion. Accordingly, in some embodiments, when an individual is determined to be experiencing spontaneous preterm labor or early term labor or spontaneous loss of pregnancy, the individual is administered estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, pregnenolone sulfate, a derivative thereof, or a combination thereof. In some embodiments the individual is administered an alternative steroidal compound of estriol-16-glucuronide, THDOC, androstane-3,17-diol, DHEA-S, a derivative thereof, or a combination thereof. In some embodiments, the individual is additionally administered progesterone, 17-α-hydroxyprogesterone, or a derivative thereof.

Administration of compounds described herein can be combined with standards of care for the complication. When an individual is experiencing spontaneous preterm labor, in some embodiments, an individual is additionally administered betamethasone, progesterone, 17-α-hydroxyprogesterone, antibiotics, magnesium sulfate, or a combination thereof. When an individual is experiencing spontaneous preterm labor, in some embodiments, an individual is administered is additionally administered at least one other tocolytic drug. Tocolytic drugs include (but are not limited to) indomethacin, orciprenaline, ritodrine, terbutaline, salbutamol, nifedipine, fenoterol, nylidrin, or isoxsuprine.

Recurrent preterm birth is a condition in which a woman experiences two or more pregnancies that go into labor prior to week 37 of gestation. Recurrent pregnancy loss is a condition in which a woman experiences two or more spontaneous losses of pregnancy. Each of these conditions are related to repeatedly experiencing uterine wall contractions occurring prematurely during gestational progress. Further, newly acquired data demonstrates that various metabolic compounds are involved in maintaining proper gestational time course and preventing uterine wall contractions and thus can be utilized in as part of treatment to mitigate spontaneous preterm labor and/or spontaneous abortion. Accordingly, in some embodiments, when an individual is diagnosed with recurrent preterm birth or recurrent pregnancy loss, the individual is administered estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, pregnenolone sulfate, a derivative thereof, or a combination thereof. In some embodiments the individual is administered an alternative steroidal compound of estriol-16-glucuronide, THDOC, androstane-3,17-diol, DHEA-S, a derivative thereof, or a combination thereof. In some embodiments, the individual is additionally administered progesterone, 17-α-hydroxyprogesterone, or a derivative thereof. In some embodiments, a treatment plan is created prior to or early after conception to prepare for the potential of preterm labor or preterm abortion.

Administration of compounds described herein can be combined with standards of care for the complication. When an individual is diagnosed with recurrent preterm birth, in some embodiments, an individual is additionally administered betamethasone, progesterone, 17-α-hydroxyprogesterone, antibiotics, magnesium sulfate, or a combination thereof. When an individual is diagnosed with recurrent pregnancy loss, in some embodiments, an individual is administered is additionally administered at least one other tocolytic drug. Tocolytic drugs include (but are not limited to) indomethacin, orciprenaline, ritodrine, terbutaline, salbutamol, nifedipine, fenoterol, nylidrin, or isoxsuprine. When an individual is experiencing recurrent pregnancy loss, in some embodiments, an individual is additionally administered a compound in combination with progesterone, 17-α-hydroxyprogesterone, human menopausal gonadotropin, a derivative thereof, or a combination thereof.

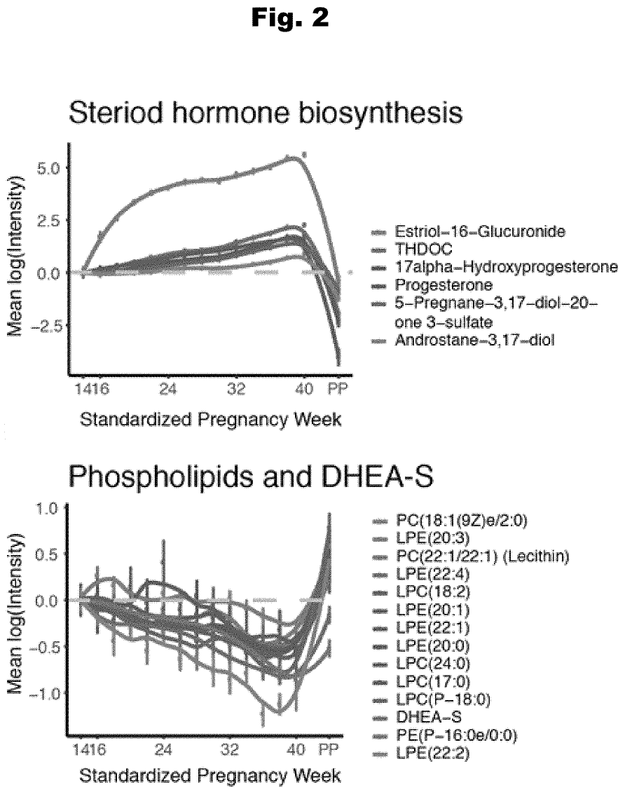

Several embodiments are directed towards metabolite surveillance during gestation. As shown in the examples provided, a number of metabolites (especially steroidal hormones) are correlate with the gestational progress. For instance, steroidal hormones estriol-16-glucuronide, THDOC, 17-α-hydroxyprogesterone, progesterone, 5-pregnane-3,7-diol-20-one-3-sulfate, and androstane-3,17-diol each steadily increase as gestation progresses towards delivery and then drop sharply to promote uterine contractions and labor (see FIG. 2). Accordingly, various metabolites help maintain an appropriate gestational timeline and prevent premature uterine contractions. In some embodiments, the concentrations and/or balance of metabolites are monitored to ensure they are at a requisite concentration and/or balance. In some embodiments, if an individual's metabolites drop below a requisite concentration and/or balance, the individual is administered a metabolite to correct the imbalance.

An exemplary method to monitor metabolites is provided:
Gather metabolite samples from an individual periodically during gestation
Measure metabolite concentrations
If a metabolite concentration is below a requisite amount or in imbalance, administer to the individual the metabolite

In some embodiments, a biological sample (e.g., blood, plasma, vaginal swab, urine, saliva or other appropriate sample) is extracted from an individual to determine the concentration of one or more of the following metabolites: estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, pregnenolone sulfate, progesterone, or 17-α-hydroxyprogesterone. In some embodiments, as part of monitoring method, an individual with a deficiency in a particular metabolite can be administered that metabolite. In some embodiments, the metabolite that is deficient and to be administered is estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, pregnenolone sulfate, progesterone, 17-α-hydroxyprogesterone, a derivative thereof, or a combination thereof.

Monitoring and reconstitution methods can be performed on any pregnant individual. In some embodiments, the individual to be monitored is at risk for spontaneous preterm labor and/or spontaneous abortion. In some embodiments, the individual has been diagnosed with recurrent preterm birth and/or recurrent pregnancy loss. In some embodiments, the individual has a family history of recurrent preterm birth and/or recurrent pregnancy loss. In some embodiments, the individual is generally healthy or has no known medical issues related to gestation.

In various embodiment, an individual is administered a compound (for example, estriol-16-glucuronide, THDOC, androstane-3,17-diol, DHEA-S, a derivative thereof, or a combination thereof), for a period of 2 to 4 weeks, for a period of 4 to 6 weeks, for a period of 6 to 8 weeks, for a period of 8 to 10 weeks, for a period of 10 to 12 weeks, for a period of 12 to 14 weeks, for a period of 14 to 19 weeks, for a period of 20 weeks, for a period of 21 weeks, for a period of 22 weeks, for a period of 23 weeks, for a period of 25 weeks, for a period of 26 weeks, for a period of 27 weeks, for a period of 28 weeks, for a period of 29 weeks, for a period of 30 weeks, for a period of 35 weeks, for a period of 37 weeks, for a period of 38 weeks, for a period of 39 weeks, for a period of 40 weeks, or for a period of more than 50 weeks.

In various embodiments, the effective amount of a compound (for example estriol-16-glucuronide, THDOC, androstane-3,17-diol, DHEA-S, a derivative thereof, or a combination thereof) is 0.5-1 mg/day, 1-5 mg/day, 5-10 mg/day, 10-15 mg/day, 15-20 mg/day, 20-25 mg/day, 25-30 mg/day, 30-35 mg/day, 35-40 mg/day, 40-45 mg/day, 45-50 mg/day, 50-55 mg/day, 55-60 mg/day, 60-65 mg/day, 65-70 mg/day, 70-75 mg/day, 75-80 mg/day, 80-85 mg/day, 85-90 mg/day, 90-95 mg/day or 95-100 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day, 2900-3000 mg/day, 3000-3100 mg/day, 3100-3200 mg/day, 3200-3300 mg/day, 3300-3400 mg/day, 3400-3500 mg/day, 3500-3600 mg/day, 3600-3700 mg/day, 3700-3800 mg/day, 3800-3900 mg/day, 3900-4000 mg/day, 4000-4200 mg/day, 4200-4400 mg/day, 4400-4600 mg/day, 4600-4800 mg/day or 4800-5000 mg/day.

### EXEMPLARY EMBODIMENTS

Biological data support the methods of treating menstrual and gestational complications. In the ensuing sections, exemplary methods and exemplary experiments performed related to uterine contraction and gestational progress (i.e., gestational age and/or time to delivery) are provided indicating that various compounds can be utilized to treating individual having a menstrual or gestational complication.

### Example 1: Correlation of Various Metabolites with Human Pregnancy

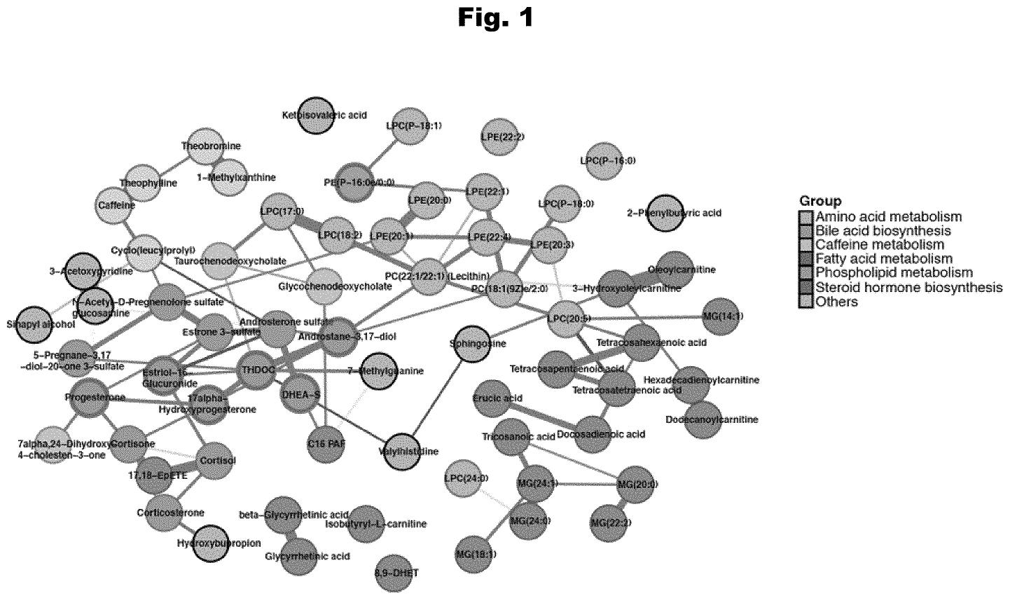

In one experiment, a cohort of pregnant woman had their metabolites from weekly blood extractions analyzed and measured. Based on the measured dynamics, a regularized partial correlation network was built (FIG. 1). Regularized partial correlation network determines critical mechanistic relationships between the identified compounds, and not just a superficial/nominal correlation. Accordingly, the identified network provides functional meaning.

The compounds that reside in the center of the partial correlation network also provide strong prediction of gestational progress (see WO2020/061590; see also L. Liang, et al., Cell. 2020; 181(7):1680-1692.e15; the disclosures of which are each incorporated herein by reference). Therefore, these central compounds are within the core of the hormone regulatory mechanisms of gestation and controlling gestation progression (as already understood to be true for progesterone). As uterine contraction and menstruation have related hormone regulation, the use of these compounds can be extrapolated to be involved in the regulation of uterine contraction and/or menstruation.

In addition, these compounds demonstrate strong mutual correlation with progesterone levels in blood, and also connected widely with other metabolites with broad physiological functions, such as lipids (FIGS. 1 and 2). In addition, a portion of these metabolites (steroids) are structurally related (but also distinct) to progesterone. It was also noted that many of the detected steroids are precursor or derivatives of progesterone or estradiol, and thus exist within similarly defined metabolic pathways. These results provide further evidence that these compounds are regulators of the gestation progress.

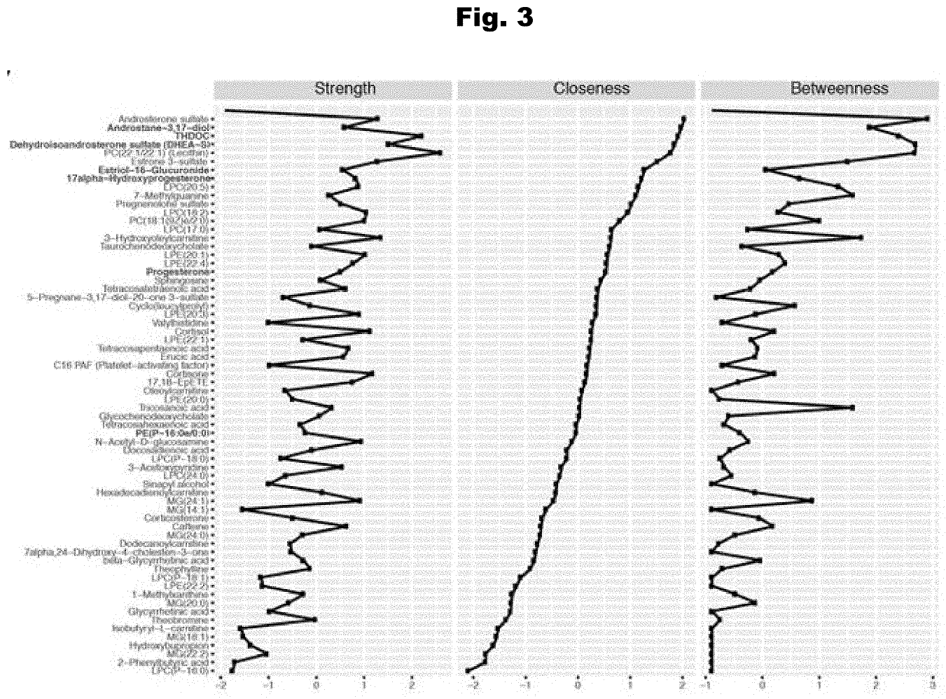

These identified compounds have high closeness ranking in the regularized partial correlation network, and also show high correlation with gestational age and low variation (FIGS. 2 and 3). Many of these compounds are likely to be more important regulators of gestational progress than progesterone, as indicated by their higher rank in the closeness score (FIG. 3). In addition, many of these compounds provide higher contribution in predicting gestational age than progesterone (see WO2020/061590; see also L. Liang, et al., Cell. 2020, cited supra).

### Example 2: Contraction of Human Uterine Smooth Muscle Cells

The abilities of various hormonal steroids to inhibit human uterine smooth muscle cell contraction were assessed utilizing a collagen gel contraction assay. Collagen (150 μl) was aliquoted per well of a 48-well plate. After 1 hour of polymerization, 80,000 human uterine smooth muscle cells (SMCs), were added to each well in 300 μl of SmBM smooth muscle basic medium (Lonza) with vehicle (DMSO) or compounds (2.5 μM of DHEA-S, 10 μM of THDOC, 10 μM of estriol-16-glucuronide, 10 μM of androstane-3,17-diol, or 2 mM of progesterone). Collagen gels were exposed to oxytocin (100 nM) to stimulate uterine SMC contraction or PBS as control. After incubating for 1 hour at 37° C. and 5% CO₂, the sides of the collagen gels were detached from the wells with sterile 200-μl pipette tips. After 18 hours, cells in collagen-coated wells were fixed with 4% paraformaldehyde solution for 30 min, then rinsed, and stored in PBS. Experiments were done multiple times (n=7), with duplicates for each condition. Surface area of each gel circle was measured using ImageJ. The areas were then expressed as a percentage relative to the area of no cell (collagen alone) control (set at 100%).

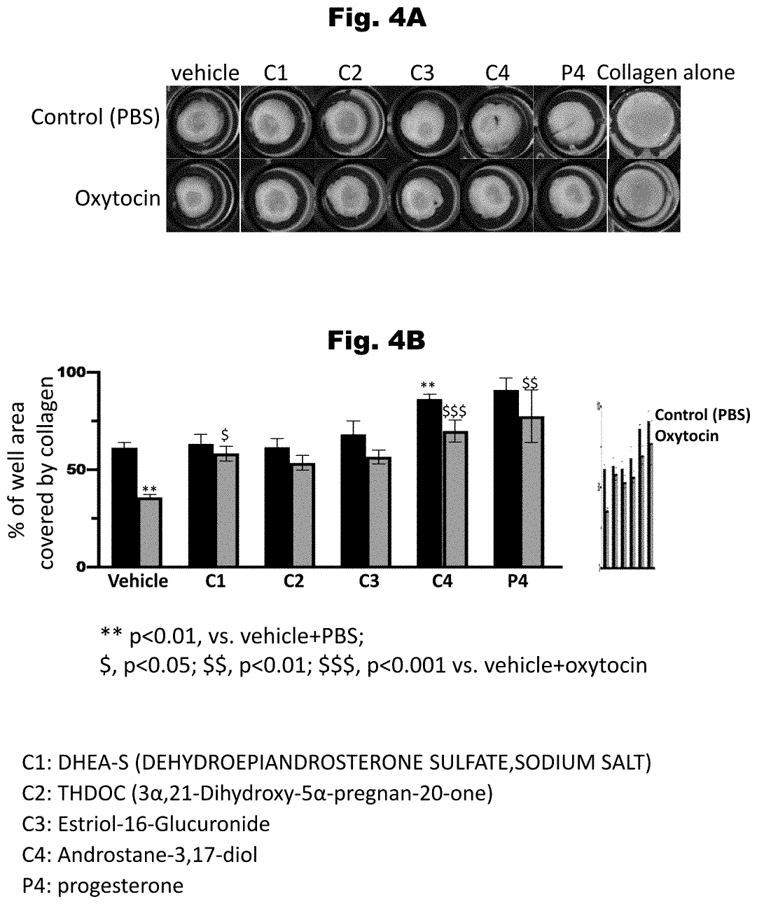

Representative image results are provided in FIG. 4A. Graphical results are provided in FIG. 4B. Each of the compounds assessed provided some ability to mitigate collagen retraction. Androstane-3,17-diol (at 10 μM) provided the best results, and slightly better than progesterone at 200 times the concentration (at 2 mM; no effect of progesterone at the same concentration as androstane-3,17-diol). These results suggest that these compounds prevent human uterine cells from contracting.

### Example 3: Contraction of Mouse Uterine Muscle Tissue

Uterine muscle strips were extracted from pregnant mice and were assessed for contractibility via myography. Timed-pregnant wild-type C57/B6 mice were euthanized before making a vertical midline abdominal incision and removing each of the two uterine horns. Uterine horns were subsequently cut into 3-mm×7-mm segments and placed in a modified Krebs buffer (118 mM NaCl, 4.8 M KCl, 1.2 mM MgSO₄, 1.2 mM KH₂PO₄, 2.5 mM CaCl₂), 25 mM NaHCO₃, and 11 mM glucose, pH 7.4). Muscle segments were vertically suspended on custom-made stainless steel hooks inside an organ bath filled with modified Krebs buffer, maintained at 37° C., and aerated with gas containing 95% O₂ and 5% CO₂. The strips were allowed to equilibrate at 1.5 g of tension for 45 min. All uterine strips demonstrated a spontaneous contraction pattern.

For the oxytocin dose-response experiments, the strips were then stimulated with oxytocin at concentrations ranging from 1 to 1000 nM, and the pattern of contraction in response to each dose was recorded for 9 min. The muscle strips were washed with Krebs solution twice and allowed to equilibrate for 3 min before the next stimulation. In separate experiments, strips were treated with the compounds before assessing the dose response to oxytocin. Contraction tracings were recorded using PowerLab and LabChart (ADInstruments).

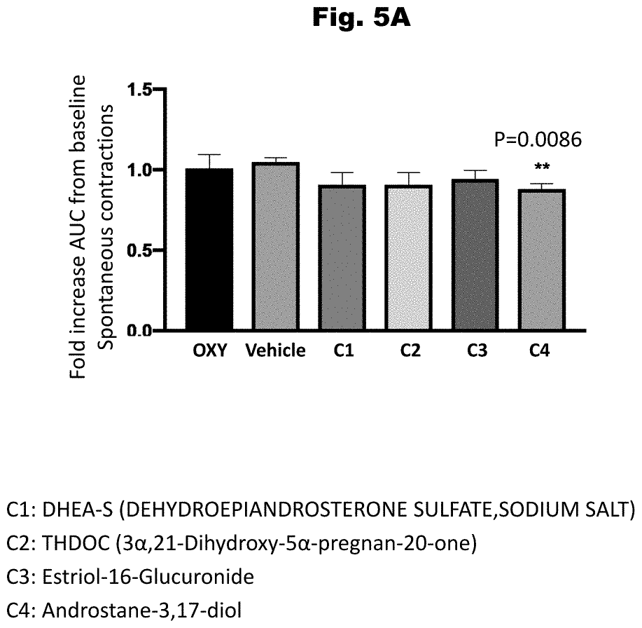

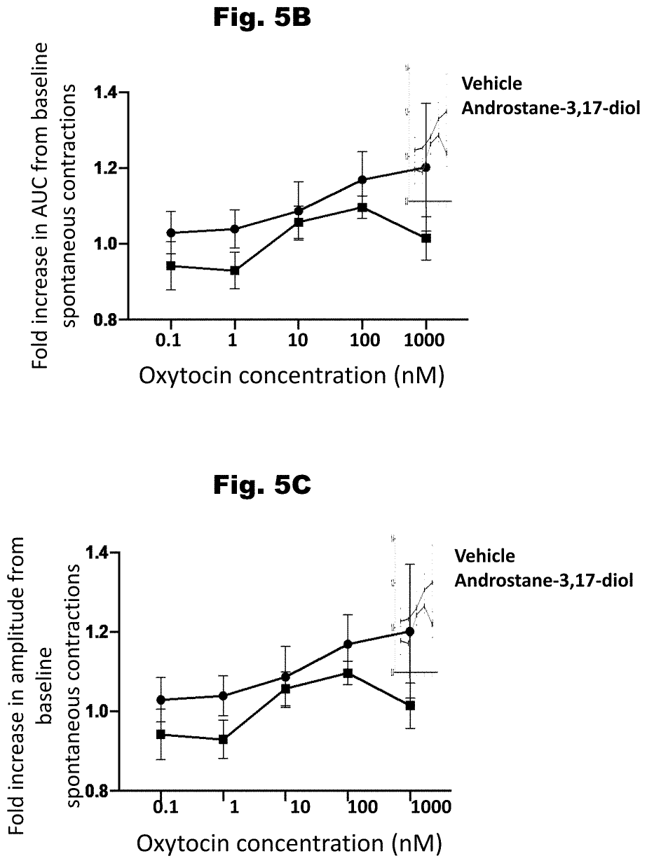

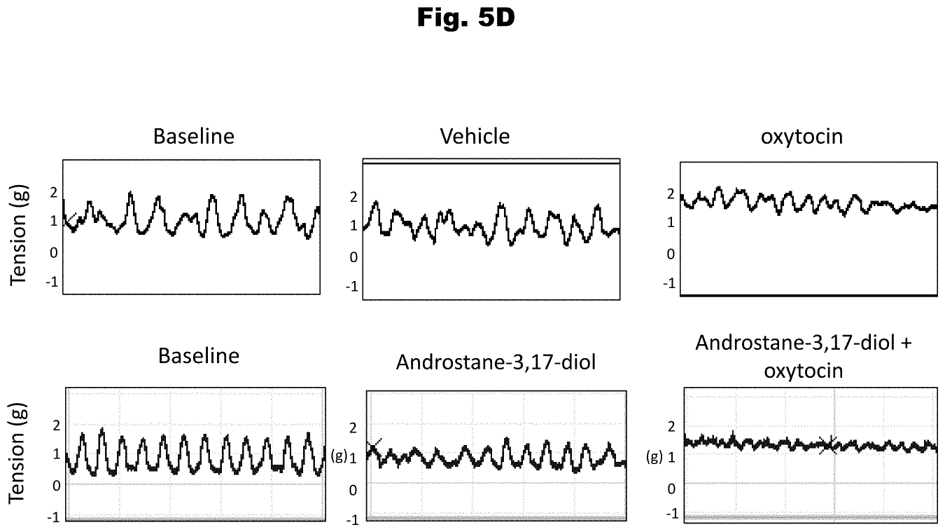

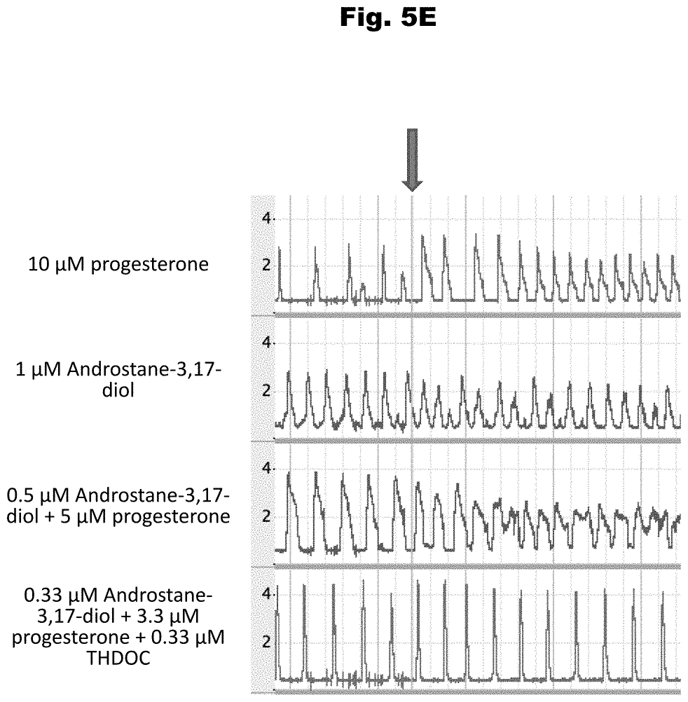

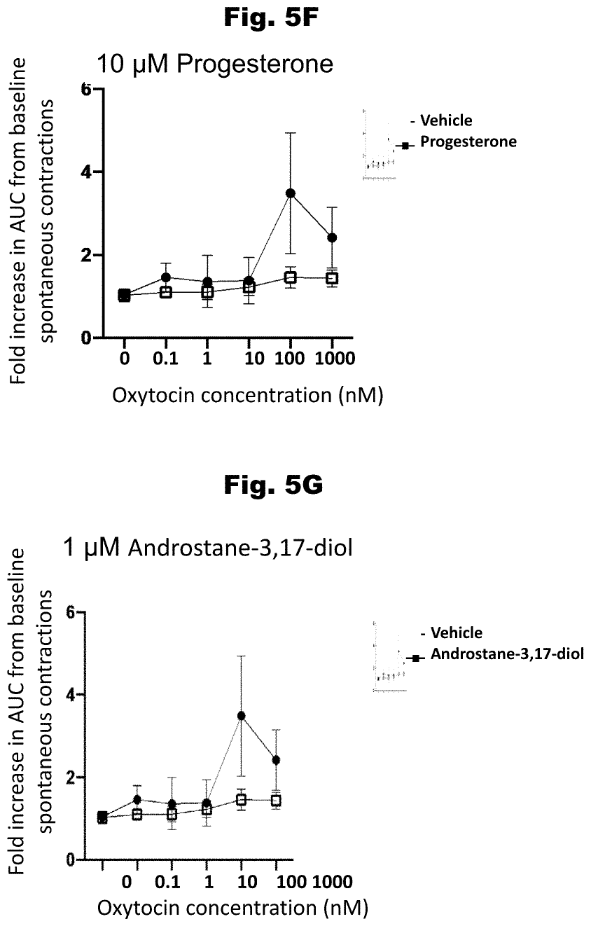

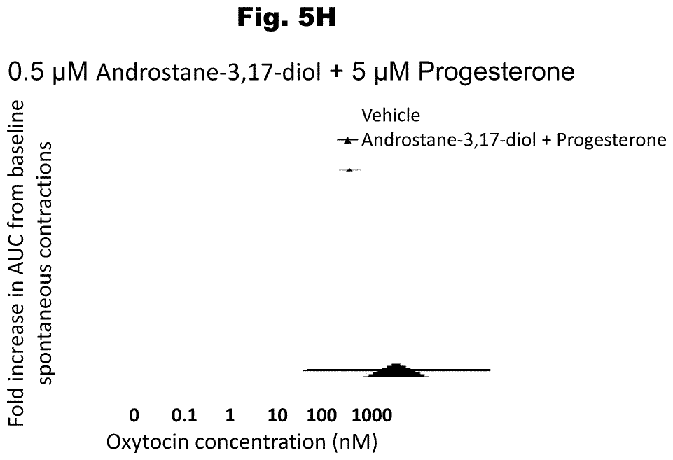

Provided in FIG. 5A are results of a myography experiment measuring spontaneous contraction. Myography of uterine muscle strips from pregnant WT mice at D18 was performed while incubating the strips with oxytocin (OXY), Vehicle, 0.25 μM μM of DHEA-S, 1 μM of THDOC, 1 μM of estriol-16-glucuronide, or 1 μM of androstane-3,17-diol. The area under the curve was calculated for the final 180 s of each 9-min contraction tracing using GraphPad Prism (GraphPad Prism for Macintosh version 5.0; GraphPad Software). The area under the curve for each response was then normalized to the area under the curve of the baseline. Compounds showed suppression of uterine contractility compared with controls, with androstane-3,17-diol showing the most significant inhibition effect.

Provided in FIGS. 5B and 5C are results of a myography experiment measuring spontaneous contractions induced by oxytocin. Myography of uterine muscle strips from pregnant WT mice at D18 was performed while incubating the strips with increasing doses of oxytocin in the presence of 1 μM Androstane-3,17-diol (black squares) or vehicle (black circles), n=4 to 7. The results are provided as old increase in area under the curve AUC (FIG. 5B), and amplitude (FIG. 5C) from baseline spontaneous contractions with vehicle and Androstane-3,17-diol. Oxytocin induced a dose-dependent increase in uterine contractility (increased in AUC but slightly decreased in amplitude) that was reduced by Androstane-3,17-diol. FIG. 5D provides representative myography tracings of baseline (lower left; upper left), response to 1 μM Androstane-3,17-diol alone (lower middle), 1 μM Androstane-3,17-diol with 1000 nM oxytocin stimulation (lower right), vehicle alone, (upper middle), or vehicle with 1000 nM oxytocin stimulation (upper right).

Provided in FIG. 5E are myography tracings recorded with various compound applications: 10 μM progesterone, 1 μM Androstane-3,17-diol, 0.5 μM Androstane-3,17-diol+5 μM progesterone, and 0.33 μM Androstane-3,17-diol+0.33 μM THDOC+3.3 μM progesterone. Left of the arrow is baseline contraction and to the right of the arrow indicates when the compounds were applied. When treated with 10 μM progesterone, the muscle contractility seemed to be first enhanced and then gradually suppressed in peak height (no effects were seen when treated with 1 μM progesterone). By contrast, 1 μM Androstane-3,17-diol provided a rapid inhibition of the muscle contraction. Intriguingly, combining 0.5 μM Androstane-3,17-diol with 5 μM progesterone had a stronger inhibition to the muscle contractility, while combing 0.33 μM Androstane-3,17-diol with 3.3 μM progesterone and 0.33 μM THDOC only produced a marginal inhibitory effect. Note that there was no obvious effect when treating the muscle with 1 μM progesterone (data not shown).

Provided in FIGS. 5F to 5H are results of a myography experiment measuring spontaneous contractions induced by oxytocin. Myography of uterine muscle strips from pregnant WT mice at D18 was performed while incubating the strips with increasing doses of oxytocin in the presence of vehicle compared with 10 μM progesterone (FIG. 5F), 1 μM Androstane-3,17-diol (FIG. 5G), and a combination of 0.5 μM androstane-3,17-diol with 5 μM progesterone (FIG. 5H), n=3. Androstane-3,17-diol but progesterone inhibits uterine muscle contraction with oxytocin stimulation. Oxytocin induced a dose-dependent increase in uterine contractility. The combination of Androstane-3,17-diol and progesterone strongly blocked the contraction even with oxytocin stimulation (C), providing a synergistic effect.

### Example 4: Compound Treatment in Mouse Models of Preterm Birth

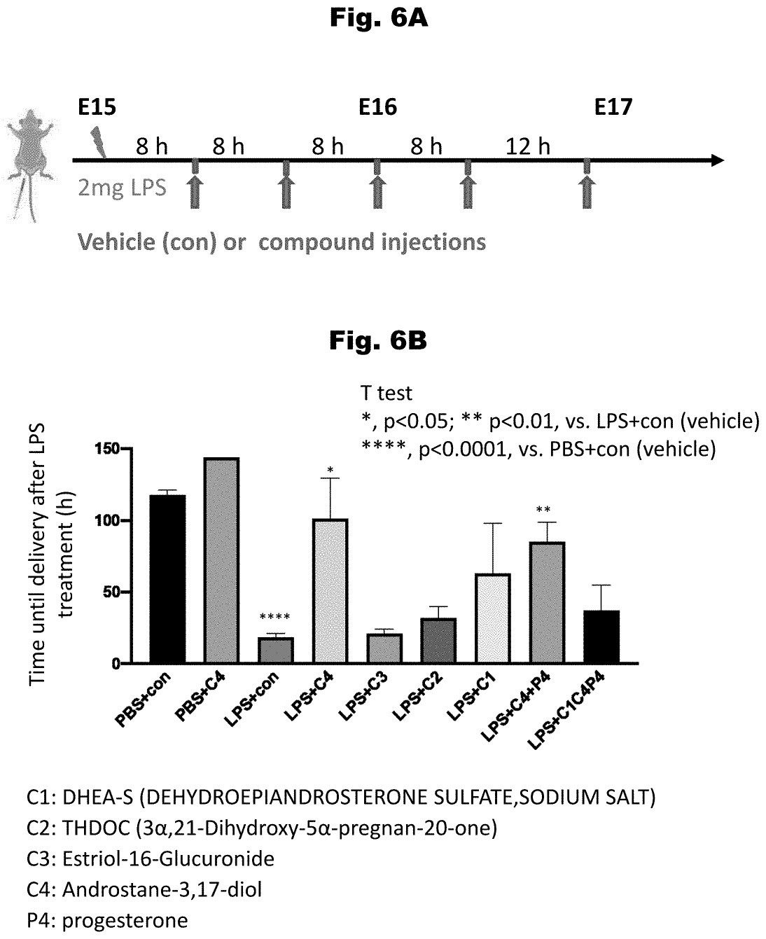

Provided in FIGS. 6A and 6B are an experimental schematic and results of compound treatment in mouse models of moderate preterm birth. Pregnant C57BL/6 mice were intraperitoneally administered 2 mg/kg LPS (Escherichia coli 0127:B8, chromatographically pure, Sigma-Aldrich) or PBS (as control) on D15. Mice were then treated with either intraperitoneal injections of vehicle (100 μl, 30% DMSO in pharmaceutical sesame oil), DHEA-S(first two doses at 0.8 mg/mouse, later doses at 0.4 mg/mouse, 100 μl), THDOC (first two doses at 0.8 mg/mouse, later doses at 0.4 mg/mouse, 100 μl), estriol-16-glucuronide (first two doses at 0.8 mg/mouse, later doses at 0.4 mg/mouse, 100 μl), androstane-3,17-diol (1.5 mg/mouse, 100 μl), Androstane-3,17-diol+progesterone (each at 0.75 mg/mouse, 100 μl), or DHEA-S+androstane-3,17-diol+progesterone (androstane-3,17-diol and progesterone each at 0.75 mg/mouse, DHEA-S at 0.27 mg/mouse for the first two doses and then 0.13 mg/mouse for the later does, 100 μl). Compound groups received the compounds via intraperitoneal injections every 8 hours, beginning 8 hours after LPS administration, up to 4 doses, and then after 12 hours before delivery. The results suggest DHEA-S, THDOC, androstane-3,17-diol, androstane-3,17-diol+progesterone combined, or DHEA-S+androstane-3,17-diol+progesterone combined prolonged gestation in vivo. Androstane-3,17-diol+progesterone combined and androstane-3,17-diol alone provided the most significant results. Full-term live pups were birthed from preterm model females treated with DHEA-S, androstane-3,17-diol, or androstane-3,17-diol+progesterone combined.

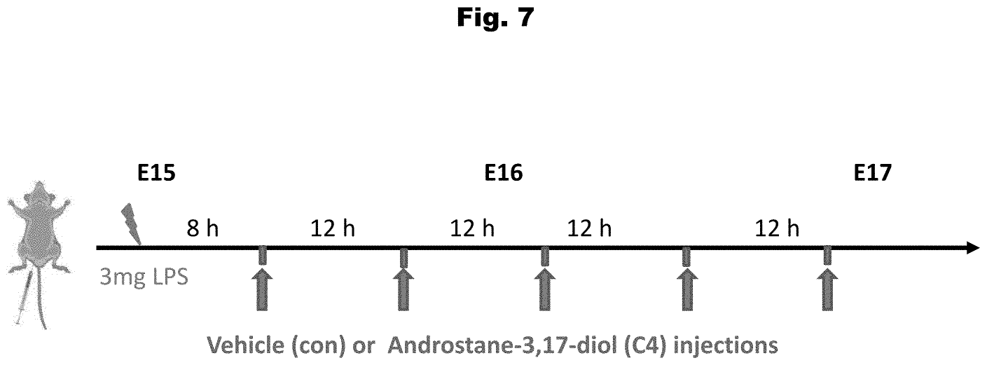

Provided in FIG. 7 is an experimental schematic of compound treatment in mouse models of severe preterm birth. In separate experiments, pregnant C57BL/6 mice were intraperitoneally administered 3 mg/kg LPS (Escherichia coli 0127:B8, chromatographically pure, Sigma-Aldrich) or PBS (as control) on D15. Treated mice were then treated with either intraperitoneal injections of vehicle (200 μl, 15% DMSO in pharmaceutical sesame oil) or androstane-3,17-diol (1.5 mg/mouse, 200 μl) every 12 hours, up to 5 doses before delivery. All LPS treated female mice that received vehicle (n=2) delivered within 32 hours after LPS treatment (i.e., preterm), while 1/3 mice receiving androstane-3,17-diol treatments after LPS reached full-term, with both mom and pups looked healthy.

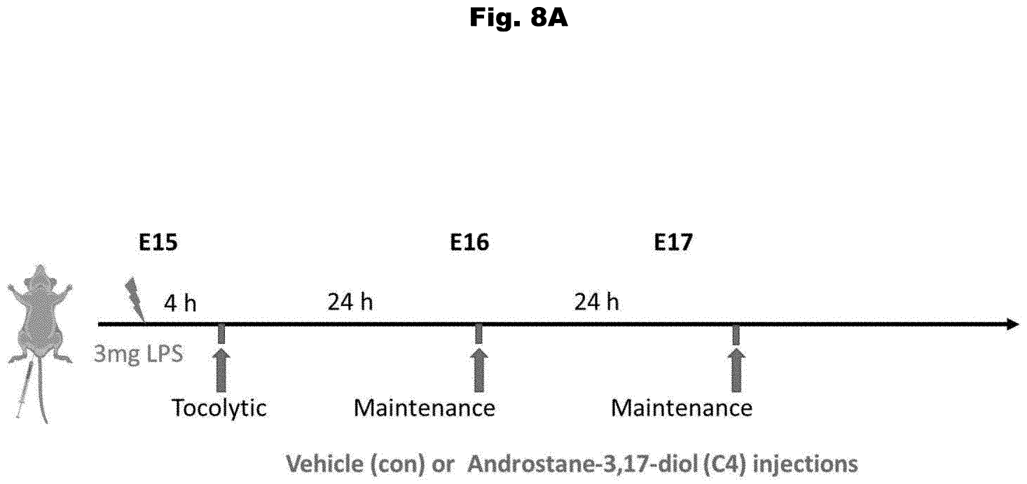

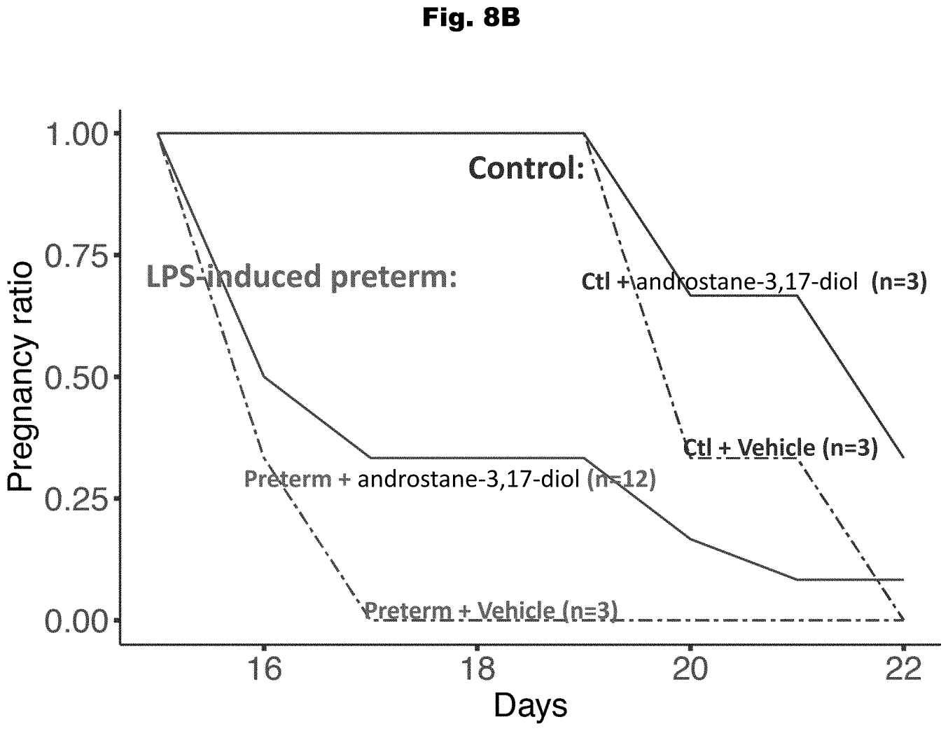

Provided in FIGS. 8A and 8B are an experimental schematic and results of tocolytic compound treatment in mouse models of severe preterm birth. In separate experiments, pregnant C57BL/6 mice were intraperitoneally administered 3 mg/kg LPS (Escherichia coli 0127:B8, chromatographically pure, Sigma-Aldrich) or PBS (as control) on D15. Treated mice were then treated with either intraperitoneal injections of vehicle or androstane-3,17-diol (1.5 mg/mouse). The androstane-3,17-diol group received C4 via intraperitoneal injections every 24 hours, up to 3 days before delivery. One-third (33%) of LPS-induced preterm mice treated with androstane-3,17-diol reached full term. Interestingly, androstane-3,17-diol treatment on healthy control mice (i.e., no LPS administration) increased gestation period beyond the typical date of delivery. These results provide that androstane-3,17-diol mitigates preterm delivery and extends the gestation period.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method of treating a pregnant individual for recurrent preterm birth, recurrent early term birth, or recurrent pregnancy loss, comprising:
determining or having determined that a pregnant individual has been diagnosed with recurrent preterm birth, recurrent early term birth, or recurrent pregnancy loss;
monitoring the individual during the individual's gestation; and
administering to the individual at least one compound to mitigate early term birth, preterm birth or pregnancy loss, wherein the at least one compound is: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, dehydroisoandrosterone sulfate (DHEA-S) or an alternative steroidal compound thereof or a derivative thereof or a metabolite within the synthesis pathway thereof, estrone 3-sulfate, N-Acetyl-D-glucosamine, 3-acetoxypyridine, 5-pregnane-3,7-diol-20-one-3-sulfate, androsterone, PC(22:1/22:1) (Lecithin), LPC(20:5), 7-methylguanine, androsterone sulfate, PE(P-16:0e/0:0) (LysoPE(P-16:0/0:0), 1-(1Z-hexadecenyl)-sn-glycero-3-phosphoethanolamine, or pregnenolone sulfate.

2. The method of claim 1, wherein at least two of the following compounds are administered to the individual: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, dehydroisoandrosterone sulfate (DHEA-S) or an alternative steroidal compound thereof or a derivative thereof or a metabolite within the synthesis pathway thereof, estrone 3-sulfate, N-Acetyl-D-glucosamine, 3-acetoxypyridine, 5-pregnane-3,7-diol-20-one-3-sulfate, androsterone, PC(22:1/22:1) (Lecithin), LPC(20:5), 7-methylguanine, androsterone sulfate, PE(P-16:0e/0:0) (LysoPE(P-16:0/0:0), 1-(1Z-hexadecenyl)-sn-glycero-3-phosphoethanolamine, or pregnenolone sulfate.

3. The method of claim 1, wherein the alternative steroidal compound of estriol-16-glucuronide is estradiol 17β-D-glucuronide.

4. The method of claim 1, wherein the alternative steroidal compound of tetrahydrodeoxycorticosterone (THDOC) is 5α-dihydrodeoxycorticosterone (DHDOC).

5. The method of claim 1, wherein the alternative steroidal compound of androstane-3,17-diol is oxandrolone, oxymetholone, stanozolol, norethandrolone, quinbolone, metandienone metenolone, prasterone, or stanolone.

6. The method of claim 1, wherein the derivative of androstane-3,17-diol is 17α-ethynyl-3α-androstanediol (apoptone), 17α-ethynyl-3β-androstanediol, 17α-ethynyl-5-androstenediol, 17α-ethynyl-5-androstenediol 3β-cyclohexanepropionate, 17α-ethynylestradiol, 17α-ethynyltestosterone, or 17α-ethynyldihydrotestosterone.

7. The method of claim 1, wherein the derivative of androstane-3,17-diol has a structural formula:

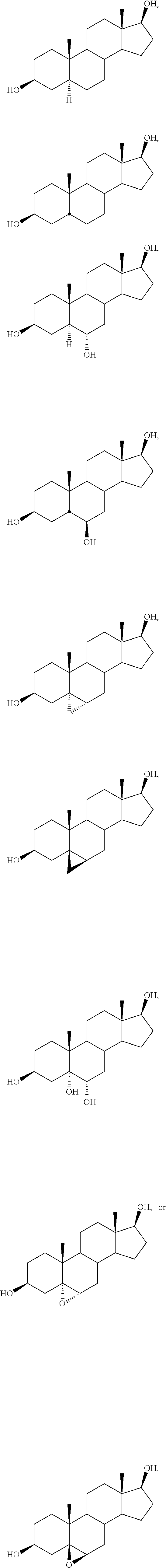

8. The method of claim 1, wherein the androstane-3,17-diol or derivative thereof has a structural formula: 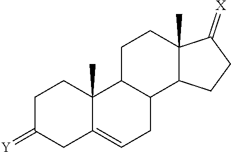
wherein X and Y are each independently: O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

9. The method of claim 1, wherein the metabolite within the synthesis pathway of androstane-3,17-diol is dehydroisoandrosterone sulfate (DHEA-S), 4-androstene-3-17-dione (androstenedione; 4A), testosterone, 5α-dihydrotestosterone (5α-DHT), 5β-dihydrotestosterone (5β-DHT), DHEA (dehydroepiandrosterone), or androsterone.

10. The method of claim 1, wherein the alternative steroidal compound dehydroisoandrosterone sulfate (DHEA-S) is 7α-hydroxy-DHEA, 16α-hydroxy-DHEA, 17α-hydroxypregnenolone, norethandrolone, oxandrolone, quinbolone, oxymetholone, metenolone, metandienone, stanozolol, and stanolone.

11. The method of claim 1, wherein the derivative of dehydroisoandrosterone sulfate (DHEA-S) is 3β-dehydroxy-16α-fluoro-DHEA (fluasterone).

12. The method of claim 1, wherein the derivative of dehydroisoandrosterone sulfate (DHEA-S) has a structural formula:

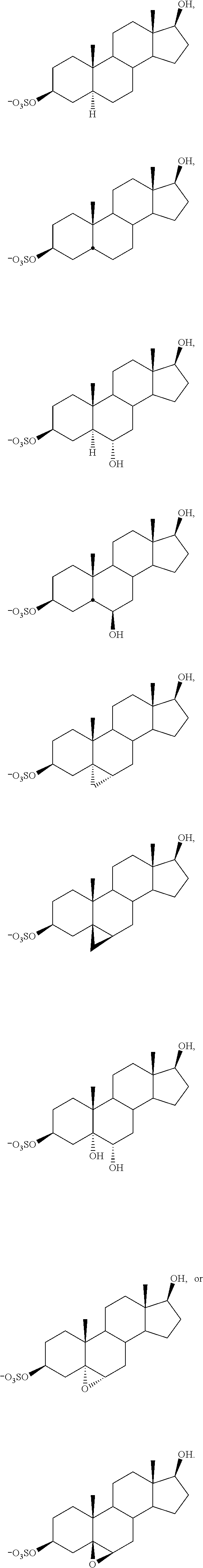

13. The method of claim 1, wherein the dehydroisoandrosterone sulfate (DHEA-S) or derivative thereof has a structural formula: 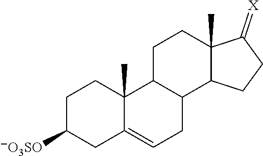
wherein X is O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

14. The method of claim 1, wherein the metabolite within the synthesis pathway of dehydroisoandrosterone sulfate (DHEA-S) is 4-androstene-3-17-dione (androstenedione; 4A), testosterone, 5α-dihydrotestosterone (5α-DHT), 5β-dihydrotestosterone (5β-DHT), DHEA (dehydroepiandrosterone), androsterone, and androstane-3,17-diol.

15. The method of any one of claims 1 to 14 further comprising administering progesterone, 17-α-hydroxyprogesterone, 17-α-hydroxyprogesterone caproate, or a progestin to the individual.

16. The method of any one of claims 1 to 15 further comprising:
extracting or having extracted a biological sample from the individual;
determining or having determined that the individual has deficiency of at least one following metabolites: estriol-16-glucuronide, tetrahydrodeoxycorticosterone (THDOC), androsterone sulfate, PE(P-16:0e/0:0) (LysoPE(P-16:0/0:0), 1-(1Z-hexadecenyl)-sn-glycero-3-phosphoethanolamine, estrone 3-sulfate, N-Acetyl-D-glucosamine, 3-acetoxypyridine, 5-pregnane-3,7-diol-20-one-3-sulfate, androsterone, androstane-3,17-diol, dehydroisoandrosterone sulfate (DHEA-S), PC(22:1/22:1) (Lecithin), LPC(20:5), 7-methylguanine, or pregnenolone sulfate;
wherein the compound that is administered to the individual is the at least one deficient metabolite, an alternative steroidal compound of the at least one deficient metabolite, a derivative of the at least one deficient metabolite, or a metabolite within the synthesis pathway the at least one deficient metabolite.

17. A method of treating a pregnant individual for early term birth, spontaneous preterm birth or spontaneous abortion, comprising:
determining or having determined that a pregnant individual is experiencing early term birth, spontaneous preterm birth or spontaneous abortion; and
administering to the individual at least one tocolytic compound to mitigate uterine contractions, wherein the at least one compound is: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

18. The method of claim 17, wherein at least two of the following tocolytic compounds are administered to the individual: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

19. The method of claim 17, wherein the alternative steroidal compound of estriol-16-glucuronide is estradiol 17β-D-glucuronide.

20. The method of claim 17, wherein the alternative steroidal compound of tetrahydrodeoxycorticosterone (THDOC) is 5α-dihydrodeoxycorticosterone (DHDOC).

21. The method of claim 17, wherein the alternative steroidal compound of androstane-3,17-diol is oxandrolone, oxymetholone, stanozolol, norethandrolone, quinbolone, metandienone metenolone, prasterone, or stanolone.

22. The method of claim 17, wherein the derivative of androstane-3,17-diol is 17α-ethynyl-3α-androstanediol (apoptone), 17α-ethynyl-3β-androstanediol, 17α-ethynyl-5-androstenediol, 17α-ethynyl-5-androstenediol 3β-cyclohexanepropionate, 17α-ethynylestradiol, 17α-ethynyltestosterone, or 17α-ethynyldihydrotestosterone.

23. The method of claim 17, wherein the derivative of androstane-3,17-diol has a structural formula:

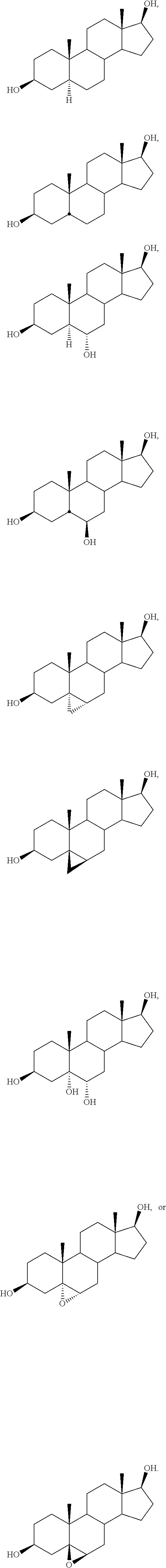

24. The method of claim 17, wherein the androstane-3,17-diol or derivative thereof has a structural formula: 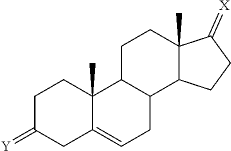
wherein X and Y are each independently: O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

25. The method of claim 17, wherein the metabolite within the synthesis pathway of androstane-3,17-diol is dehydroisoandrosterone sulfate (DHEA-S), 4-androstene-3-17-dione (androstenedione; 4A), testosterone, 5α-dihydrotestosterone (5α-DHT), 5β-dihydrotestosterone (5β-DHT), DHEA (dehydroepiandrosterone), or androsterone.

26. The method of claim 17, wherein the alternative steroidal compound dehydroisoandrosterone sulfate (DHEA-S) is 7α-hydroxy-DHEA, 16α-hydroxy-DHEA, 17α-hydroxypregnenolone, norethandrolone, oxandrolone, quinbolone, oxymetholone, metenolone, metandienone, stanozolol, and stanolone.

27. The method of claim 17, wherein the derivative of dehydroisoandrosterone sulfate (DHEA-S) is 3β-dehydroxy-16α-fluoro-DHEA (fluasterone).

28. The method of claim 17, wherein the derivative of dehydroisoandrosterone sulfate (DHEA-S) has a structural formula:

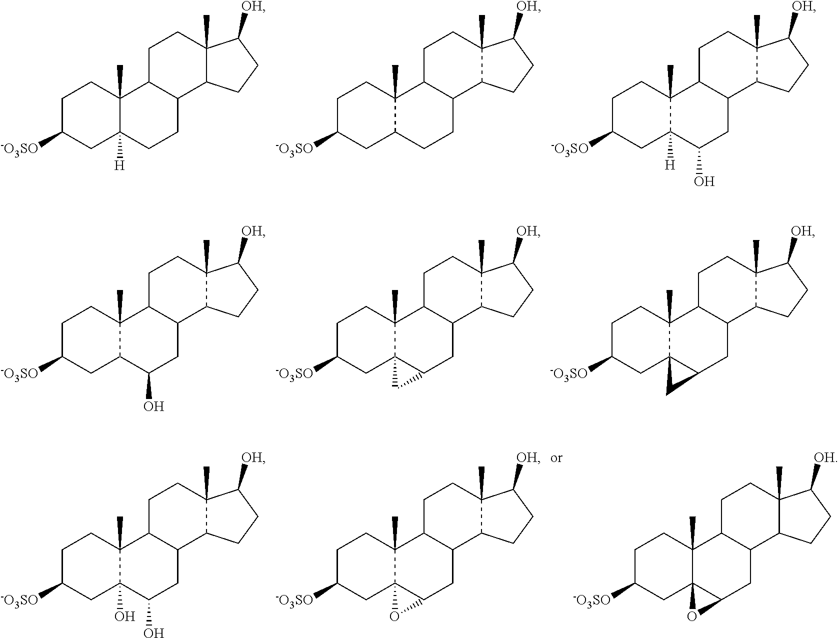

29. The method of claim 17, wherein the dehydroisoandrosterone sulfate (DHEA-S) or derivative thereof has a structural formula: 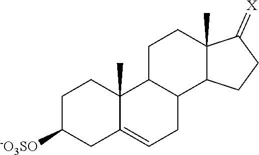
wherein X is O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

30. The method of claim 17, wherein the metabolite within the synthesis pathway of dehydroisoandrosterone sulfate (DHEA-S) is 4-androstene-3-17-dione (androstenedione; 4A), testosterone, 5α-dihydrotestosterone (5α-DHT), 5β-dihydrotestosterone (5β-DHT), DHEA (dehydroepiandrosterone), androsterone, and androstane-3,17-diol.

31. The method of any one of claims 17 to 30 further comprising administering progesterone, 17-α-hydroxyprogesterone, 17-α-hydroxyprogesterone caproate, or a progestin to the individual.

32. The method of any one of claims 17 to 31 further comprising:
extracting or having extracted a biological sample from the individual;
determining or having determined that the individual has deficiency of at least one following metabolites: estriol-16-glucuronide, tetrahydrodeoxycorticosterone (THDOC), androstane-3,17-diol, or dehydroisoandrosterone sulfate (DHEA-S);
wherein the compound that is administered to the individual is the at least one deficient metabolite, an alternative steroidal compound of the at least one deficient metabolite, a derivative of the at least one deficient metabolite, or a metabolite within the synthesis pathway the at least one deficient metabolite.

33. A method of treating an individual for menorrhagia or dysmenorrhea, comprising:
determining or having determined that an individual is diagnosed with menorrhagia or dysmenorrhea; and
administering to the individual at least one compound to mitigate menorrhagia or dysmenorrhea, wherein the at least one compound is: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

34. The method of claim 33, wherein at least two of the following compounds are administered to the individual: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

35. The method of claim 33, wherein the alternative steroidal compound of estriol-16-glucuronide is estradiol 17β-D-glucuronide.

36. The method of claim 33, wherein the alternative steroidal compound of tetrahydrodeoxycorticosterone (THDOC) is 5α-dihydrodeoxycorticosterone (DHDOC).

37. The method of claim 33, wherein the alternative steroidal compound of androstane-3,17-diol is oxandrolone, oxymetholone, stanozolol, norethandrolone, quinbolone, metandienone metenolone, prasterone, or stanolone.

38. The method of claim 33, wherein the derivative of androstane-3,17-diol is 17α-ethynyl-3α-androstanediol (apoptone), 17α-ethynyl-3β-androstanediol, 17α-ethynyl-5-androstenediol, 17α-ethynyl-5-androstenediol 3β-cyclohexanepropionate, 17α-ethynylestradiol, 17α-ethynyltestosterone, or 17α-ethynyldihydrotestosterone.

39. The method of claim 33, wherein the derivative of androstane-3,17-diol has a structural formula:

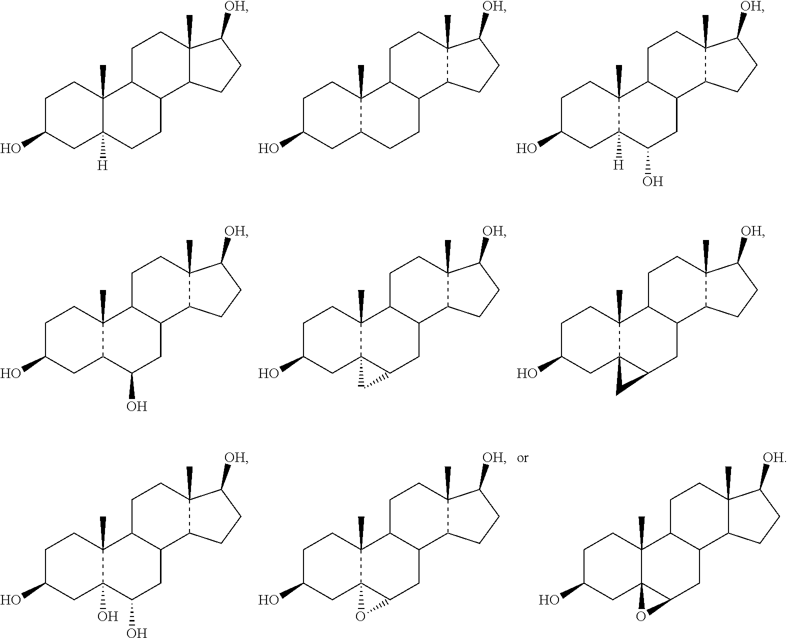

40. The method of claim 33, wherein the androstane-3,17-diol or derivative thereof has a structural formula: 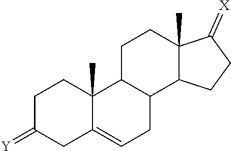
wherein X and Y are each independently: O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

41. The method of claim 33, wherein the metabolite within the synthesis pathway of androstane-3,17-diol is dehydroisoandrosterone sulfate (DHEA-S), 4-androstene-3-17-dione (androstenedione; 4A), testosterone, 5α-dihydrotestosterone (5α-DHT), 5β-dihydrotestosterone (5β-DHT), DHEA (dehydroepiandrosterone), or androsterone.

42. The method of claim 33, wherein the alternative steroidal compound dehydroisoandrosterone sulfate (DHEA-S) is 7α-hydroxy-DHEA, 16α-hydroxy-DHEA, 17α-hydroxypregnenolone, norethandrolone, oxandrolone, quinbolone, oxymetholone, metenolone, metandienone, stanozolol, and stanolone.

43. The method of claim 33, wherein the derivative of dehydroisoandrosterone sulfate (DHEA-S) is 3β-dehydroxy-16α-fluoro-DHEA (fluasterone).

44. The method of claim 33, wherein the derivative of dehydroisoandrosterone sulfate (DHEA-S) has a structural formula:

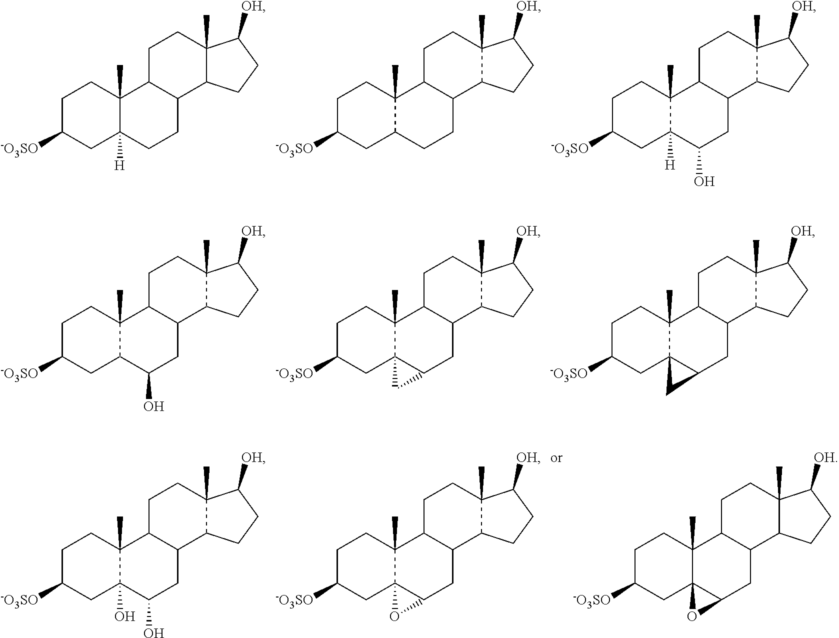

45. The method of claim 33, wherein the dehydroisoandrosterone sulfate (DHEA-S) or derivative thereof has a structural formula: 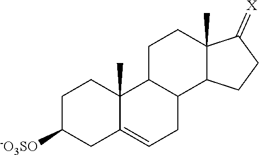
wherein X is O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

46. The method of claim 33, wherein the metabolite within the synthesis pathway of dehydroisoandrosterone sulfate (DHEA-S) is 4-androstene-3-17-dione (androstenedione; 4A), testosterone, 5α-dihydrotestosterone (5α-DHT), 5β-dihydrotestosterone (5β-DHT), DHEA (dehydroepiandrosterone), androsterone, and androstane-3,17-diol.

47. The method of any one of claims 33 to 46 further comprising administering progesterone, 17-α-hydroxyprogesterone, 17-α-hydroxyprogesterone caproate, or a progestin to the individual.

48. The method of any one of claims 33 to 47 further comprising:
extracting or having extracted a biological sample from the individual;
determining or having determined that the individual has deficiency of at least one following metabolites: estriol-16-glucuronide, tetrahydrodeoxycorticosterone (THDOC), androstane-3,17-diol, or dehydroisoandrosterone sulfate (DHEA-S);
wherein the compound that is administered to the individual is the at least one deficient metabolite, an alternative steroidal compound of the at least one deficient metabolite, a derivative of the at least one deficient metabolite, or a metabolite within the synthesis pathway the at least one deficient metabolite.

49. A method of treating a pregnant individual to prolong gestation, comprising:
determining or having determined that an individual is pregnant; and
prior to the individual having uterine contractions associated with neonatal delivery, administering to the individual at least one compound to prolong gestation, wherein the at least one compound is: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

50. The method of claim 49, wherein at least two of the following compounds are administered to the individual: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

51. The method of claim 49, wherein the alternative steroidal compound of estriol-16-glucuronide is estradiol 17β-D-glucuronide.

52. The method of claim 49, wherein the alternative steroidal compound of tetrahydrodeoxycorticosterone (THDOC) is 5α-dihydrodeoxycorticosterone (DHDOC).

53. The method of claim 49, wherein the alternative steroidal compound of androstane-3,17-diol is oxandrolone, oxymetholone, stanozolol, norethandrolone, quinbolone, metandienone metenolone, prasterone, or stanolone.

54. The method of claim 49, wherein the derivative of androstane-3,17-diol is 17α-ethynyl-3α-androstanediol (apoptone), 17α-ethynyl-3β-androstanediol, 17α-ethynyl-5-androstenediol, 17α-ethynyl-5-androstenediol 3β-cyclohexanepropionate, 17α-ethynylestradiol, 17α-ethynyltestosterone, or 17α-ethynyldihydrotestosterone.

55. The method of claim 49, wherein the derivative of androstane-3,17-diol has a structural formula:

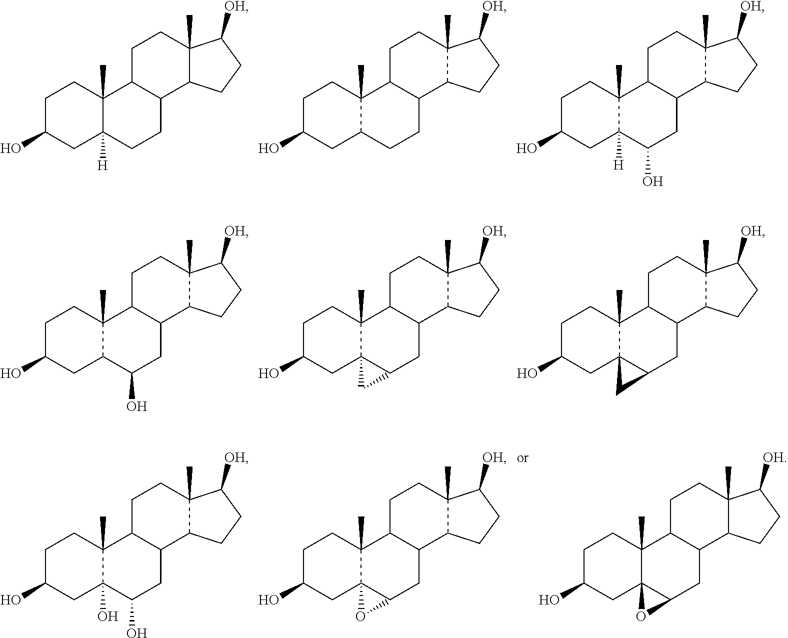

56. The method of claim 49, wherein the androstane-3,17-diol or derivative thereof has a structural formula: 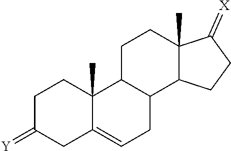
wherein X and Y are each independently: O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

57. The method of claim 49, wherein the metabolite within the synthesis pathway of androstane-3,17-diol is dehydroisoandrosterone sulfate (DHEA-S), 4-androstene-3-17-dione (androstenedione; 4A), testosterone, 5α-dihydrotestosterone (5α-DHT), 5β-dihydrotestosterone (5β-DHT), DHEA (dehydroepiandrosterone), or androsterone.

58. The method of claim 49, wherein the alternative steroidal compound dehydroisoandrosterone sulfate (DHEA-S) is 7α-hydroxy-DHEA, 16α-hydroxy-DHEA, 17α-hydroxypregnenolone, norethandrolone, oxandrolone, quinbolone, oxymetholone, metenolone, metandienone, stanozolol, and stanolone.

59. The method of claim 49, wherein the derivative of dehydroisoandrosterone sulfate (DHEA-S) is 3β-dehydroxy-16α-fluoro-DHEA (fluasterone).

60. The method of claim 49, wherein the derivative of dehydroisoandrosterone sulfate (DHEA-S) has a structural formula:

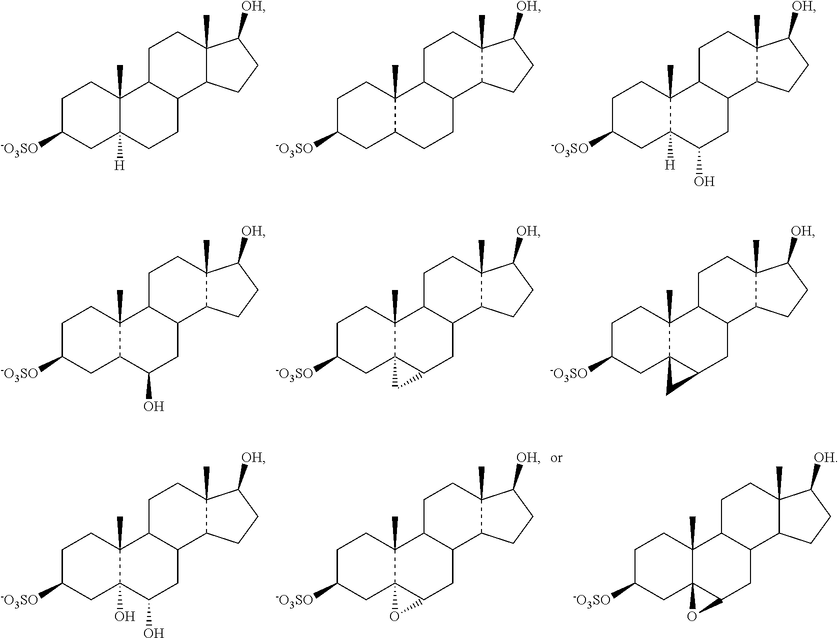

61. The method of claim 49, wherein the dehydroisoandrosterone sulfate (DHEA-S) or derivative thereof has a structural formula: 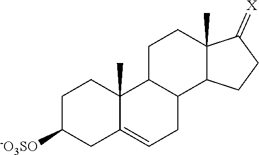
wherein X is O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

62. The method of claim 49, wherein the metabolite within the synthesis pathway of dehydroisoandrosterone sulfate (DHEA-S) is 4-androstene-3-17-dione (androstenedione; 4A), testosterone, 5α-dihydrotestosterone (5α-DHT), 5β-dihydrotestosterone (5β-DHT), DHEA (dehydroepiandrosterone), androsterone, and androstane-3,17-diol.

63. The method of any one of claims 49 to 62 further comprising administering progesterone, 17-α-hydroxyprogesterone, 17-α-hydroxyprogesterone caproate, or a progestin to the individual.

64. The method of any one of claims 49 to 63, wherein the individual is generally healthy or has no known medical issues related to gestation.

65. A medicament for use in mitigating uterine contractions in an individual, the medicament comprising:
estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

66. The medicament of claim 65, wherein the medicament comprises at least two of the following compounds: estriol-16-glucuronide or an alternative steroidal compound thereof, tetrahydrodeoxycorticosterone (THDOC) or an alternative steroidal compound thereof, androstatne-3,17-diol or an alternative steroidal compound thereof or a derivative thereof or a metabolite within the synthesis pathway thereof, or dehydroisoandrosterone sulfate (DHEA-S) or an alternative steroidal compound thereof or a derivative thereof or a metabolite within the synthesis pathway thereof.

67. The method of claim 65, wherein the alternative steroidal compound of estriol-16-glucuronide is estradiol 17β-D-glucuronide.

68. The medicament of claim 65, wherein the alternative steroidal compound of tetrahydrodeoxycorticosterone (THDOC) is 5α-dihydrodeoxycorticosterone (DHDOC).

69. The medicament of claim 65, wherein the alternative steroidal compound of androstane-3,17-diol is oxandrolone, oxymetholone, stanozolol, norethandrolone, quinbolone, metandienone metenolone, prasterone, or stanolone.

70. The medicament of claim 65, wherein the derivative of androstane-3,17-diol is 17α-ethynyl-3α-androstanediol (apoptone), 17α-ethynyl-3β-androstanediol, 17α-ethynyl-5-androstenediol, 17α-ethynyl-5-androstenediol 3β-cyclohexanepropionate, 17α-ethynylestradiol, 17α-ethynyltestosterone, or 17α-ethynyldihydrotestosterone.

71. The medicament of claim 65, wherein the derivative of androstane-3,17-diol has a structural formula:

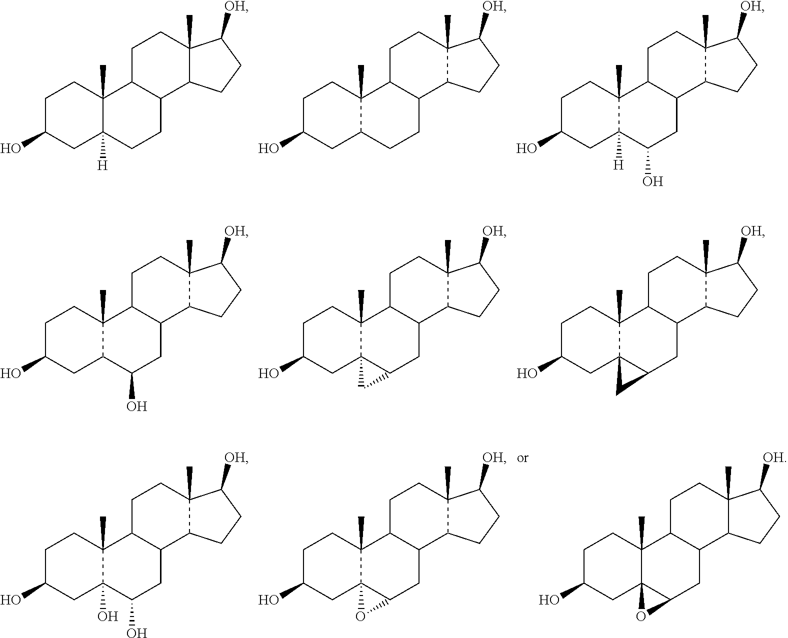

72. The medicament of claim 65, wherein the androstane-3,17-diol or derivative thereof has a structural formula: 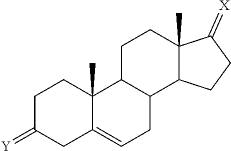
wherein X and Y are each independently: 0, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

73. The medicament of claim 65, wherein the metabolite within the synthesis pathway of androstane-3,17-diol is dehydroisoandrosterone sulfate (DHEA-S), 4-androstene-3-17-dione (androstenedione; 4A), testosterone, 5α-dihydrotestosterone (5α-DHT), 5β-dihydrotestosterone (5β-DHT), DHEA (dehydroepiandrosterone), or androsterone.

74. The medicament of claim 65, wherein the alternative steroidal compound dehydroisoandrosterone sulfate (DHEA-S) is 7α-hydroxy-DHEA, 16α-hydroxy-DHEA, 17α-hydroxypregnenolone, norethandrolone, oxandrolone, quinbolone, oxymetholone, metenolone, metandienone, stanozolol, and stanolone.

75. The medicament of claim 65, wherein the derivative of dehydroisoandrosterone sulfate (DHEA-S) is 3β-dehydroxy-16α-fluoro-DHEA (fluasterone).

76. The medicament of claim 65, wherein the derivative of dehydroisoandrosterone sulfate (DHEA-S) has a structural formula:

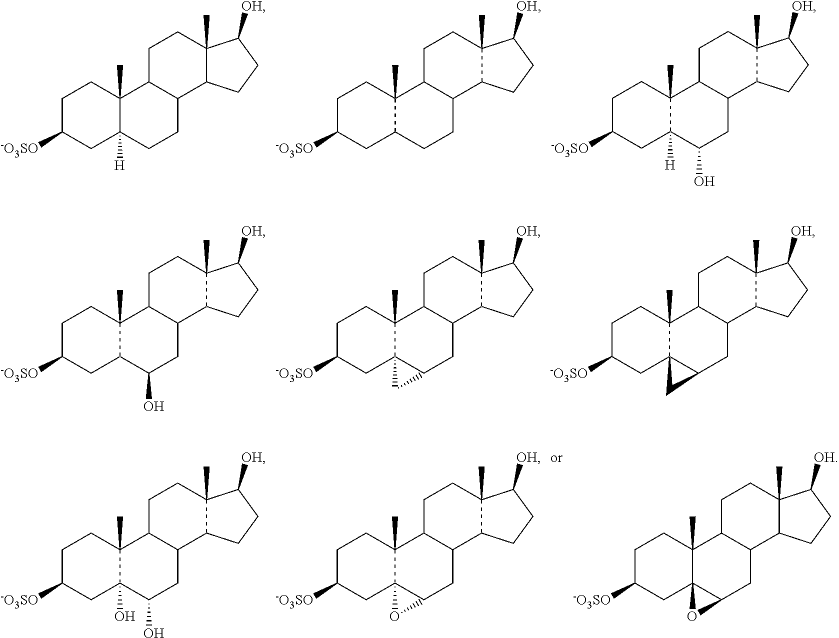

77. The medicament of claim 65, wherein the dehydroisoandrosterone sulfate (DHEA-S) or derivative thereof has a structural formula: 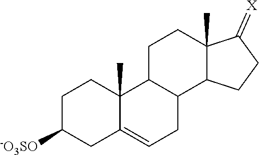
wherein X is O, NR, NOR, NNR¹R², OR⁴ α/R³ α, OR⁴ β/R³ β, —O(CH₂)ₙO—, or —O(CHR)ₙO—;
R, R², R³, and R⁴ are each independently: H, alkyl, alkenyl, alkynyl, aryl, or heteroaryl; and
n is 2, 3, or 4.

78. The medicament of claim 65, wherein the metabolite within the synthesis pathway of dehydroisoandrosterone sulfate (DHEA-S) is 4-androstene-3-17-dione (androstenedione; 4A), testosterone, 5α-dihydrotestosterone (5α-DHT), 5β-dihydrotestosterone (5β-DHT), DHEA (dehydroepiandrosterone), androsterone, and androstane-3,17-diol.

79. The medicament of claim any one of claims 65 to 78 further comprising progesterone, 17-α-hydroxyprogesterone, 17-α-hydroxyprogesterone caproate, or a progestin to the individual.

80. The medicament of claim any one of claims 65 to 78, wherein the medicament is for treating recurrent preterm birth, recurrent early term birth, or recurrent pregnancy loss.

81. The medicament of claim any one of claims 65 to 78, wherein the medicament is a tocolytic for treating early term birth, spontaneous preterm birth, or spontaneous abortion in a pregnant individual.

82. The medicament of claim any one of claims 65 to 78, wherein the medicament is for treating menorrhagia or dysmenorrhea.

83. The medicament of claim any one of claims 65 to 78, wherein the medicament is for prolonging gestation of a pregnant individual.

84. The medicament of claim 83, wherein the pregnant individual is generally healthy or has no known medical issues related to gestation.
